(19) United States
(12) Patent Application Publication
(10) Pub. No.: US 2003/0113740 A1
(43) Pub. Date: Jun. 19, 2003

(54) Oligonucleotide-modified ROMP polymers and co-polymers

(76) Inventors: Chad A. Mirkin, Wilmette, IL (US); SonBinh T. Nguyen, Evanston, IL (US); Keith J. Watson, Midland, MI (US); So-Jung Park, Evanston, IL (US)

Correspondence Address: Emily Miao, McDonnell Boehnen Hulbert & Berghoff, 32nd Floor, 300 S. Wacker Drive, Chicago, IL, 60606, US

(21) Appl. No.: 10/125,194

(22) Filed: Apr. 18, 2002

Related U.S. Application Data
(60) Provisional application No. 60/286,615, filed on Apr. 26, 2001.

(51) Int. Cl. C12Q 1/68; C08G 63/91; C08G 63/48

(52) U.S. Cl. 435/6; 525/54.2

(57) ABSTRACT

Ring-opening metathesis polymerization (ROMP) polymers or copolymers having oligonucleotides bound thereto, materials comprised of the oligonucleotide-modified ROMP polymers, and methods of making and using the same for preparing new materials and for detection of target nucleic acids are disclosed.

### CROSS-REFERENCE

This application claims the benefit of U.S. provisional application no. U.S. Prov. No. 60/286,615, filed Apr. 26, 2001, which is incorporated by reference in its entirety. The invention was supported, in part, by U.S. government agency grants by AFOSR, ARO, and NSF. Accordingly, the U.S. Government may have certain rights to the invention.

### FIELD OF THE INVENTION

This application relates to ROMP polymers or co-polymers having oligonucleotides attached thereto, materials comprising the ROMP polymer or copolymer conjugates, and methods for preparing and using the same.

### BACKGROUND OF THE INVENTION

The use of DNA as an interconnect for the synthesis of new materials with preconceived architectural parameters and properties is a field of research that has seen considerable growth over the past several years.¹ The unique and reversible recognition properties of these biomolecules are the key elements through which their utility is derived. Exploring DNA for this purpose already has led to the development of new detection strategies,² novel structures,³⁻⁸ and the construction of nanoelectronic structures.⁹ In recent years, the coupling of synthetic oligonucleotides to organic polymers has emerged as a promising research area where the combination of properties associated with both the polymer backbone and the attached DNA can be simultaneously addressed, manipulated, and optimized to achieve a particular function. For example, the attachment of DNA to polypyrrole¹⁰⁻¹⁴ and other conducting polymers,¹⁵ either through post-polymerization modification or direct copolymerization, has led to the development of polymer-based amperometric detection methods. While interesting, these DNA/polymer hybrids are limited with respect to their degree of tailorability, ill-defined compostions, and poor solubilities and dispersities, as well as function. The synthesis of well-defined block copolymer hybrids which can overcome these limitations would be an important contribution to this developing technology.

### SUMMARY OF THE INVENTION

The present invention relates to ROMP polymers or co-polymers having oligonucleotides bound thereto, materials or structures comprising the same, and methods of preparing and using the same. The applicants discovered that the covalent attachment of synthetic oligonucleotides to the backbone of a well-defined polymer structure derived from ring-opening metathesis polymerization (ROMP) reaction provides polymers that are useful in preparing novel materials. Attempts to incorporate DNA into ROMP polymers via polymerization of monomers including DNA strands were unsuccessful. Using the inventive approach, monomers are polymerized using any suitable metathesis catalyst. If two or more different monomers are used, the polymerization may occur in a stepwise or simultaneous manner to produce block and random co-polymers. Any suitable monomers may be used, however these monomers preferably include a reporter label and/or a functional group that would allow for post-polymerization modification of the preformed polymer template to attach oligonucleotides. Particularly preferred monomers are substituted norbornenes having reporter labels such as a UV tag or redox active ferrocenes. Post-polymerization modification of the resulting ROMP polymer template with 2-cyanoethyl diisopropyl chlorophosphoramidite allows the polymer to be easily be modified with DNA using standard solid phase techniques. DNA-modifed ROMP polymers or copolymers with various redox potentials can be prepared which have full DNA recognition properties and electrochemical properties. In addition, these polymers exhibit useful properties such as sharp melting transitions and high thermal stabilities.

For instance, the Examples below describe the ROMP polymerization of a norbornenyl-modified alcohol (2) substituted with a diphenylacetylene spacer as a UV-tag using a commercially available ruthenium-carbene catalyst. The resultant ROMP homopolymer was then modified with the chlorophosphoramidite and coupled to predefined DNA molecules using solid phase technique. The resulting DNA-modified ROMP polymers were characterized using UV-spectroscopy in combination with DNA hybridization studies. Aggregate structures comprised of polymers with complementary strands led to the formation of extended hybridization networks which precipitate reversibly from aqueous solutions, demonstrating that multiple DNA strands are attached to each individual polymer. When DNA modified polymers were exposed to a solution containing 13 nm gold particles with complementary strands attached to their surface, three dimensional aggregates of particles were formed and characterized using UV-Vis spectroscopy and transmission electron microscopy. Finally, block copolymers derived from 2 and norbornenyl-modified ferrocenes can be synthesized and coupled to DNA using this strategy. The presence of the second block did not interfere with the recognition properties of the DNA and imparted electrochemical properties that are useful in detecting for the presence or absence of a target nucleic acid or oligonucleotide.

Accordingly, the present invention provides a ROMP polymer or co-polymer having oligonucleotides bound thereto. The ROMP polymer may be a homopolymer. The ROMP co-polymer comprises a ROMP block co-polymer or random co-polymer. The ROMP block co-polymer includes multiblock co-polymer. The oligonucleotides bound to the polymer may comprise a spacer portion and a recognition portion wherein the spacer portion is bound to the ROMP polymer, and the recognition portion having a sequence that is complementary to at least one portion of the sequence of another oligonucleotide. If desired, the oligonucleotides comprise at least one type of recognition oligonucleotides, each type of recognition oligonucleotides comprising a spacer portion and a recognition portion wherein the spacer portion is attached to the ROMP polymer and the recognition portion has a sequence complementary to at least one portion of the sequence of another oligonucleotide. The spacer portion may include from about 4 to about 30 nucleotides, preferably 10 nucleotides and most preferably about 4 nucleotides.

The ROMP polymer or co-polymer may be derived from the polymerization of at least one monomer that can be polymerized through ring-opening metathesis polymerization in the presence of a metathesis catalyst. When two or more different monomers are used, the polymerization may be stepwise to produce block co-polymers or simultaneously to produce random co-polymers. While any suitable metathesis catalyst for ROMP reactions may be used, ruthenium or osmium carbene catalysts are preferred.

A particularly preferred metathesis catalyst comprises Cl₂Ru(PCy₃)₂═CHPh or Cl₂Ru(PPh₃)₂═CHPh. While any suitable monomer may be used, the monomer is preferably a cyclic mono-olefin such as substituted norbornene. Examples of a suitable substituted norbornene include norbornenyl-modified alcohol such as monomer 2 which include a UV tag and a norbornenyl group modified with an electrochemical tag such as a norbornenyl-modified ferrocene.

In another embodiment of the invention, a ROMP polymer is provided that comprises an oligonucleotide-modified product produced by the ROMP polymerization of monomer 2 to produce a homopolymer template and post-polymerization modification of the polymer template to attach oligonucleotides.

In yet another embodiment of the invention, a ROMP co-polymer having oligonucleotides bound thereto is provided and that is produced by the process of (a) sequential block ROMP polymerization of monomer 2 and at least one or more different monomers to produce a ROMP co-polymer template; (b) post-polymerization modification of the template, followed by coupling of oligonucleotides to the modified template. The one or more different monomers may include a substituted norbornene such as a norbornenyl group modified with an electrochemical tag, e.g a norbornenyl-substituted ferrocene.

The present invention also provides materials or structures comprising a ROMP polymer or co-polymer having oligonucleotides bound thereto. Thus, in one embodiment of the invention, materials or structures are provided that comprise a first and second ROMP polymers or copolymers having oligonucleotides bound thereto, the oligonucleotides bound to the first ROMP polymer or co-polymer having a sequence that is complementary to the oligonucleotides bound to the second ROMP polymer or co-polymer.

In yet another embodiment of the invention, materials or structures are provided that are comprised of:

(a) particles having oligonucleotides attached thereto; and

(b) a connector for holding the particles together, the connector comprising a ROMP polymer or co-polymer having oligonucleotides bound thereto, the oligonucleotides bound to the ROMP polymer or co-polymer having a sequence complementary to at least a portion of the sequence of the oligonucleotides bound to the particles. The oligonucleotides bound to the particles may have a spacer portion for attaching the oligonucleotides to the particles and a recognition portion that has a sequence that is complementary to at least a portion of the sequence of another oligonucleotide.

In yet another embodiment of the invention, materials or structures are provided that are comprised of:

(a) particles having oligonucleotides attached thereto, the oligonucleotides comprising at least one type of recognition oligonucleotides, each type of recognition oligonucleotides comprising a spacer portion and a recognition portion, the spacer portion having a functional group through which the spacer portion is bound to the particles, the recognition portion having a sequence complementary to at least one portion of the sequence of another oligonucleotide; and

(b) a connector for holding the particles together, the connector comprising a ROMP polymer or co-polymer having oligonucleotides bound thereto, the oligonucleotides comprise a spacer portion and a recognition portion, wherein the spacer portion is bound to the ROMP polymer or co-polymer and the recognition portion has a sequence complementary to at least one portion of the sequence of the oligonucleotides bound to the particles.

In yet another embodiment of the invention, materials or structures are provided that comprise:

(a) particles having oligonucleotides attached thereto, the oligonucleotides comprising:

(i) at least one type of recognition oligonucleotides, each type of recognition oligonucleotides comprising a spacer portion and a recognition portion, the spacer portion having a functional group through which the spacer portion is bound to the particles, the recognition portion having a sequence complementary to at least one portion of the sequence of another oligonucleotide; and

(ii) a type of diluent oligonucleotides; and

(b) a connector for holding the particles together, the connector comprising a ROMP polymer or co-polymer having oligonucleotides bound thereto, the oligonucleotides comprise a spacer portion and a recognition portion, wherein the spacer portion is bound to the ROMP polymer or co-polymer and the recognition portion having a sequence complementary to at least one portion of the sequence of the oligonucleotides bound to the particles.

In another embodiment of the invention, materials or structures are provided that comprise:

(a) at least two types of particles having oligonucleotides attached thereto, the first type of particle having at least two types of oligonucleotides, the first type of oligonucleotides bound to the first type of particles having a sequence that is complementary to at least a portion of the sequence of the oligonucleotides bound to a second type of particle; and

(b) oligonucleotide polymer conjugates for holding the particles together, the oligonucleotide polymer conjugate comprising a ROMP polymer having oligonucleotides bound thereto, the oligonucleotides of the oligonucleotide polymer conjugate having a sequence complementary to at least one portion of the sequence of a second type of oligonucleotides bound to the first type of particles.

The particles in the materials or structures comprise metallic particles, semiconductor particles, polymer latex particles, inorganic particles or a combination thereof. The metallic particles may be made of gold, and the semiconductor particles may be made of CdSe/ZnS (core/shell). The polymer latex particles may be composed of polyacrylates and the inorganic particles may be comprised of silica or metal oxide. Preferrably the particles are nanoparticles. The spacer portion of the oligonucleotides bound to the ROMP polymer or co-polymer comprises from about 4 to about 30 nucleotides, preferably about 4 nucleotides. The spacer portion of the oligonucleotides bound to the particles generally range between about 10 to about 30 nucleotides, preferably at least 10 nucleotides.

The present invention also provides methods for fabrication. In one embodiment of the invention, a method of fabrication is provided and comprises:

providing a ROMP polymer or co-polymer having at least one type of oligonucleotides bound thereto, the oligonucleotides having a selected sequence, the sequence of each type of oligonucleotide having at least two portions;

providing one or more types of particle-oligonucleotide conjugates, the oligonucleotides attached to the particles of each of the types of conjugates having a sequence complementary to the sequence of a portion of a oligonucleotide bound to the ROMP polymer or co-polymer; and

contacting the ROMP polymer or co-polymer and particle oligonupcleotide conjugates under conditions effective to allow hybridization of the oligonucleotides attached to the particles to the oligonucleotides bound to the ROMP polymer or co-polymer so that a desired material or structure is formed wherein the particles conjugates are held together by oligonucleotides bound to the ROMP polymer.

In another embodiment of the invention, a method of fabrication is provided that comprises:

providing at least two types of particle-oligonucleotide conjugates, the first type of particle-oligonucleotide conjugates have at least two types of oligonucleotides wherein the first type of oligonucleotides attached to the first type of particle-oligonucleotide conjugates has a sequence that is complementary to that of the oligonucleotides attached to the particles of the second type of conjugates, the second type of oligonucleotides attached to the particles of the first type of conjugates having a sequence that is complementary to that of the oligonucleotides attached to the particles of a second type of conjugates;

providing a ROMP polymer or co-polymer having oligonucleotides bound thereto, the oligonucleotides having a sequence that is complementary to a second type of oligonucleotides bound to the first type of particle-oligonucleotide conjugates;

contacting the first and second types of particle-oligonucleotide conjugates with the ROMP polymer or co-polymer under conditions effective to allow hybridization of the oligonucleotides on the first type of particle-oligonucleotide conjugates with the oligonucleotides on the second type of particle-oligonucleotide conjugates and on the ROMP polymer or co-polymer so that a desired material or structure is formed.

In another embodiment of the invention, a method of fabrication is provided that comprises:

providing first and second ROMP polymers or co-polymers having oligonucleotides bound thereto, the oligonucleotides bound to the first ROMP polymer or co-polymer having a sequence that is complementary to the oligonucleotides bound to the second ROMP polymer or co-polymer; and

contacting the first and second ROMP polymers or co-polymer under conditions effective to allow hybridization of the oligonucleotides on the first ROMP polymer or co-polymer with the oligonucleotides on the second ROMP polymer or co-polymer so that a desired material or structure is formed.

The present invention also provides a method for preparing a ROMP polymer or co-polymer having oligonucleotides bound thereto. The method comprises:

providing (i) a ROMP polymer or co-polymer modified with chlorophosphoramidite and (ii) oligonucleotides bound to a solid support;

contacting the chlorophosphoramidite-modified ROMP polymer with the oligonucleotides bound to a support to produce an oligonucleotide ROMP polymer conjugate bound to the support; and

cleaving the oligonucleotide-modified ROMP polymer or copolymer from the support.

The present invention also provides a method for the detection of one or more target nucleic acids in a sample, the sequence of each nucleic acid having at least two portions. Thus, in one embodiment of the invention, the method comprises:

providing one or more types of oligonucleotide-modified ROMP polymer or copolymer, the sequence of the oligonucleotides bound to each type of polymer or copolymer has at least two portions wherein at least one portion of the sequence of the oligonucleotides is complementary to first portion of a sequence of a target nucleic acid, wherein the oligonucleotides bound to one type of polymer or copolymer is different from another type, wherein each type of polymer or copolymer serves as a unique identifier for a particular target nucleic acid, and wherein the polymer or copolymer includes electrochemical labels;

providing a gold electrode surface having oligonucleotides bound thereto, the oligonucleotides that are bound to the surface have a sequence having at least two portions wherein the first portion of the oligonucleotides is complementary to a second portion of the target nucleic acid;

contacting the one or more types of oligonucleotide-modified ROMP polymer or copolymer, the gold surface, and the sample under conditions effective to allow for hybridization of the oligonucleotides bound to the polymer or copolymer with the target nucleic acids and for hybridization of the oligonucleotides bound to the surface with the target nucleic acids to form a complex on the surface in the presence of one or more target nucleic acids; and

electrochemically detecting for the presence of the complex,.

The ROMP polymer or copolymer are chemically defined and includes a defined number of electrochemical labels. Moreover, electrochemical detection may occur using cyclic voltammetry or differential pulse voltammetry. The surface may have a plurality of types of oligonucleotides attached thereto in an array to allow for the detection of multiple different nucleic acid targets. The sample may be first contacted with the surface so that one or more target nucleic acids hybridizes with complementary oligonucleotides bound to the surface and then the target nucleic acids bound to the surface is contacted with the polymer or copolymer so that at least some of the oligonucleotides bound to the polymer or copolymer hybridize with a portion of the sequence of the target nucleic acid bound to the surface. Alternatively, the polymer or copolymer is contacted with the sample so that at least some of the oligonucleotides bound to the polymer or copolymer hybridize with a portion of the sequence of the target nucleic acids; and contacting the target nucleic acids bound to the polymer or copolymer with the surface so that a portion of the sequence of the target nucleic acids bound to the polymer or copolymer hybridizes with complementary oligonucleotides bound to the surface. Alternatively, the sample, polymer or copolymer, and surface are contacted simultaneously.

In another embodiment of the detection method of the invention, signal amplification may be performed by providing a second oligonucleotide-modified ROMP polymer or copolymer, the sequence of the oligonucleotides bound to the second polymer or copolymer has at least two portions wherein at least one portion of the sequence of the oligonucleotides bound to the second polymer or copolymer is complementary to oligonucleotides bound to the first oligonucleotide-modified ROMP polymer or co-polymer; and contacting the second ROMP polymer or co-polymer with the one or more types of the first ROMP polymer or copolymer bound to the surface. Further signal amplication may be achieved by further providing a third oligonucleotide-modified ROMP polymer or copolymer, the sequence of the oligonucleotides bound to the second polymer or copolymer has at least two portions wherein at least one portion of the sequence of the oligonucleotides bound to the second polymer or copolymer is complementary to oligonucleotides bound to the first oligonucleotide-modified ROMP polymer or co-polymer; and contacting the third ROMP polymer or co-polymer with the second ROMP polymer or copolymer bound to the surface.

The present invention also provides kits for detecting one or more target nucleic acids in a sample. Thus, in one embodiment of the invention, the kit comprising at least one or more containers including one or more types of chlorophosphoramidite modified ROMP polymer or copolymer, wherein each polymer or copolymer has a different redox activity and can be used for coupling with oligonucleotides.

In another embodiment of the invention, the kit comprising at least one or more containers including one or more types of chlorophosphoramidite-modifiable ROMP polymer or copolymer, wherein each polymer or copolymer has a different redox activity and can serve as an identifier for a specific target nucleic acid.

In a yet another embodiment of the invention, the kit comprising at least one or more containers including one or more types of oligonucleotide-modified ROMP polymer or copolymer, wherein each polymer or copolymer has a different redox activity and serves as an identifier for a specific target nucleic acid.

The present invention also provides a system for detecting one or more target nucleic acids in a sample, the sequence of target nucleic acids have at least two portions, in a sample comprising

(a) one or more types of oligonucleotide-modified ROMP polymer or copolymer, wherein each polymer or copolymer has a different redox activity and serves as an identifier for a specific target nucleic acid, the oligonucleotides bound to one type of polymer or copolymer is different from another, the oligonucleotides have a sequence having at least two portions, one portion of the sequence of the oligonucleotides is complementary to a first portion of a target nucleic acid;

(b) a gold electrode surface having oligonucleotides bound thereto wherein the oligonucleotides bound to the surface has a sequence that is complementary to a second portion of a target nucleic acid; and

(c) a detector for electrochemical detection of one or more polymers or copolymers bound to the surface in the presence of one or more target nucleic acids.

These and other embodiments of the invention will be apparent in light of the detailed discussion below.

### DESCRIPTION OF THE FIGURES

FIG. 1. Synthetic scheme illustrating preparation of a DNA-modified ROMP polymer from monomer 2 in the presence of catalyst Cl₂Ru Cy₃)₂═CHPh 1, modifying the ROMP polymer (poly2) using chlorophosphoramidite 3 and coupling the modified ROMP polymer to DNA using solid phase synthesis. Two complementary DNA-modified ROMP polymers were prepared: 3′-GCG TAA GTC CTA A₁₀-5′-poly2 (Hybrid I) and 3′-TAG GAC TTA CGC A10-5′-poly2 (Hybrid II).

FIG. 2. Synthetic scheme illustrating the preparation of new monomers and intennediates in Example 1.

FIG. 3: A. UV-Vis spectrum of Hybrid-1. B. The UV-Vis spectra of Hybrid-I/Hybrid-II mixture before an after melting (melting curve inset) C. The UV-Vis spectra of DNA-modified 13-nm Au particles and aggregates of Hybrid-I and complementary DNA-modified 13-nm Au particles. D. TEM image of the aggregates from C.

FIG. 4. Synthetic scheme illustrating preparation of a DNA-modified ROMP block co-polymer from monomer 2 and monomer 4 via ROMP polymerization using catalyst 1, post-polymerization of the poly2-block-poly4 co-polymer with chlorophosphoramidite 3 and coupling the modified ROMP polymer to DNA using solid phase synthesis.

FIG. 5. (A) The cyclic voltammogram of Hybrid IV in 0.2 M [(n-Bu)₄N]PF₆ in CH₂Cl₂. (B) The melting curve for Hybrid-III/Hybrid-IV in a PBS buffer (first derivative inset).

FIG. 6. Synthetic scheme illustrating preparation of a redox-active DNA-modified ROMP block polymers from monomer 2 and a norbornenyl-modified ferrocene monomer in the presence of catalyst Cl₂Ru(PCy₃)₂═CHPh 1, modifying the resultant ROMP polymer using chlorophosphoramidite 3 and coupling the modified ROMP polymer to DNA using solid phase synthesis. Suitable, but non-limiting, examples of norbornenyl-modified ferrocene monomers are illustrated therein.

FIG. 7. A. The UV-Vis absorption spectrum of DNA/ROMP polymer hybrids in water. B. VW-Vis absorption spectrum of purified Hybrid I. One major peak at 25 min was observed at both 260 and 310 nm, indicating that DNA is coupled to polymer backbone.

FIG. 8. A cyclic voltammogram of Hybrid I (—) and III ( - - - - - ) in in 0.2 M[(Bu)₄N]PF₆ in CH₂Cl₂.

FIG. 9. Scheme illustrating examples of redox active ROMP triblock co-polymers having blocks that differ in size and type of norbornenyl-modified ferrocene monomers.

FIG. 10. DPV of (A) triblock copolymers and (B) random block copolymers.

FIG. 11. (A) UV-vis spectra of the solution containing complementary hybrid molecules (Hybrid I:Hybrid II) before and after DNA melting temperature. (B) Thermal denaturation curves of aggregates formed from hybrid molecules. A thermal denaturation curve for duplex DNA formed from oligonucleotides with same sequences as Hybrid I and II is given for comparison.

FIG. 12. DNA detection scheme using DNA-modified ROMP block copolymer probes. Target nucleic acid sequence a′b′ binds via portion a′ to the complementary oligonucleotides a that are bound to the gold electrode surface. The ROMP polymer having ferrocenes as electrochemical tags and oligonucleotides b (complementary to b′) bind to the nucleic acid.

FIG. 13. Alternating Current (AC) voltammograms illustrating that gold electrodes treated with complementary target nucleic acid sequence produced a detectable signal while no signal was detected in the absence of complementary target.

FIG. 14. Scheme illustrating the UV spectrum of oligonucleotide-modified ROMP polymer before and after Centricon-50 ultrafiltration.

FIG. 15. Scheme illustrating signal amplification of an complex of oligonucleotide-modified ROMP co-polymer, a target nucleic acid, and oligonucleotides bound to a gold electrode surface as shown in FIG. 12. A second oligonucleotide b′ (complementary to b)-modified ROMP co-polymer is hybridized to the complex to form a second complex. Thereafter, a third oligonucleotide b (complementary to b′)-modified ROMP copolymer is hybridized to the second complex.

### DETAILED DESCRIPTION OF THE INVENTION

Herein, we report the covalent attachment of DNA to the backbone of a well-defined organic polymer derived from ring-opening metathesis polymerization (ROMP) reaction. This reaction generally involves the catalyzed reaction of a cyclic olefin monomer to yield an unsaturated polyolefin or polymer:

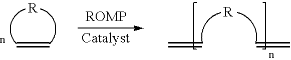

Given the thorough exploration and optimization of ROMP during the past decade,¹⁶ its use as a template for the construction of DNA/polymer hybrid materials offers several distinct advantages over other polymeric systems. The commercially available catalyst Cl₂Ru(PCy3)₂═CHPh (1) has been shown to initiate the polymerization of ring-strained olefins (such as norbornene) in a living manner and to be exceptionally tolerant to a large number of diverse functional groups. These properties have led to the isolation of heretofore unattainable polymers and block copolymers with virtually any functional group covalently attached to the polymer chain, making ROMP an ideal tool for the isolation of novel and useful materials.¹⁷ The combination of such wide ranging functionalities with the unique recognition properties of DNA could lead to the development of new materials with easily programmable parameters.

ROMP has been used to generate defined, biologically active polymers (Gibson et al., Chem. Commun., 1095-1096 (1997); Biagini et al., Chem. Commun., 1097-1098 (1997); Biagini et al., Polymer, 39, 1007-1014 (1998); and Kiessling et al., Topics in Organometallic Chemistry, 1, 199-231 (1998)) with potent and unique activities that range from inhibiting protein-carbohydrate recognition events to promoting the proteolytic release of cell surface proteins (Mortell et al., J. Am. Chem. Soc., 118, 2297-2298 (1996); Mortell et al., J. Am. Chem. Soc., 116, 12053-12054 (1994); Kanai et al., J. Am. Chem. Soc., 119, 9931-9932 (1997)); Kingsbury et al., J. Am. Chem. Soc., 121, 791-799 (1999); Schrock et al., J. Am. Chem. Soc., 112, 3875-3886 (1990); Gordon et al., Nature, 392, 30-31 (1998); and Sanders et al., J. Biol. Chem., 274, 5271-5278 (1999). In addition to these advantageous properties, ROMP polymers have a number of advantages. Specifically, the ROMP reaction can be performed under living polymerization conditions, and if the rate of initiation is faster than that of propagation, varying the monomer to initiator ratio (M:I) can generate materials of defined length (Ivin and Mol, Olefin Metathesis and Metathesis Polymerization, 2^{nd}. Ed.; Academic Press: San Diego, 1997). This approach has been successfully applied with the Grubb's ruthenium metal carbene catalyst ([(Cy)₃P]₂Cl₂Ru═CHPh) to generate materials with narrow polydispersities, indicating that the resulting substances are fairly homogeneous (Dias et al., J. Am. Chem. Soc., 119, 3887-3897 (1997); and Lynn et al., J. Am. Chem. Soc., 118, 784-790 (1996)). In contrast to anionic and cationic polymerization catalysts, ruthenium metal carbene initiators are tolerant of a wide range of functional groups.

In practicising this invention, conventional ROMP polymerization reaction conditions and any suitable metathesis catalyst may be used to prepare the ROMP polymer or co-polymers used a templates to prepare the oligonucleotide-modified ROMP polymers or copolymers. The parameters for the ROMP polymerization reactions used in the present invention, such as the atmosphere, choice of catalyst, the ratio of catalyst to monomer, the reaction temperatures, the solvents that may be used, the additives and other agents that may be present during the polymerization reaction, and the methods for carrying out the metathesis polymerization will vary and can be selected by one of ordinary skill in the art without undue experimentation. Many suitable conditions and parameters are described, for instance, in Schwab et al., J. Am. Chem. Soc., 118, 100-110 (1996) an Lynn et al., J. Am. Chem. Soc. 118, 784-790 (1996); David S. Breslow “Progress in Polymer Science” 1993, 18, pp. 1141-1195; K. J. Ivin and J. C. Mol in “Olefin Metathesis and Metathesis Polymerization,” 2^{nd} ed., Academic Press, San Diego, 1997, pp. 260-339; R. H. Grubbs and W. Tumas, Science, pp. 907-915 (Feb. 17 1989); R. R. Schrock in “Alkene Metathesis in Organic Synthesis” A. Furstner, Ed., Springer-Verlaag, 1998, pp. 1-36; L. L. Kiessling and L. E. Strong in “Alkene Metathesis in Organic Synthesis” A. Furstner, Ed., Springer-Verlaag, 1998, pp. 199-231; Warner U.S. Pat. No. 6,323,296; and Kiessling U.S. Pat. No. 6,291,616; and references cited therein, which are incorporated by reference in their entirety.

Generally the polymerization of the olefin is carried out by adding the metathesis catalyst to a solution of the monomer starting material which has been heated to an initial reaction temperature. Alternatively, the catalyst may be first added to the monomer starting material and the mixture then heated to the required temperature. The initial reaction temperature is not critical; but, as is known, this temperature does affect the rate of the polymerization reaction. Generally the reaction temperature will be in the range of about 0° C. to about 100° C., and preferably about 25° C. to about 45° C. The reaction is generally carried out under an inert atmosphere (e.g., nitrogen or argon). Pressure is not critical, but may be varied to maintain a liquid phase reaction mixture. Reaction times can vary from several minutes to several days.

The ratio of catalyst to starting material is not critical and can within the range from about 1:5 to about 1:200,000 by mole. Ratios of catalyst to starting material of between about 1:2,000 and 1:15,000 by mole are preferred. The invention may be practiced using catalyst/starting material ratios outside of the above ranges.

The monomer starting material may optionally be refluxed, either in a solution or by itself, run through absorption purification, and degassed before the catalyst is added; although, none of these procedures is necessary in practicing the invention.

Although it is preferred that the reaction be conducted in the present of solvent or mixture of solvents, the presence of a solvent is not critical. Possible solvents that may be used include organic, protic, or aqueous solvents which are inert under the reaction conditions. Examples of suitable solvents may include aromatic hydrocarbons, chlorinated hydrocarbons, ethers, alipabtic hydrocarbons, alcohols, water, etc. which are unreactive under the reaction conditions. Specific examples include 1,2-dichloroethane, benzene, toluene, p-xylene, methylene chloride, dichlorobenzene, tetrahydrofuran, diethylether, pentane, methanol.

In ROMP reactions, the polymer is generally terminated by reacting the catalyst with a capping agent. The capping agent is typically matched to the catalyst. For ruthenium catalyst, for example, ethyl vinyl ether has been used.

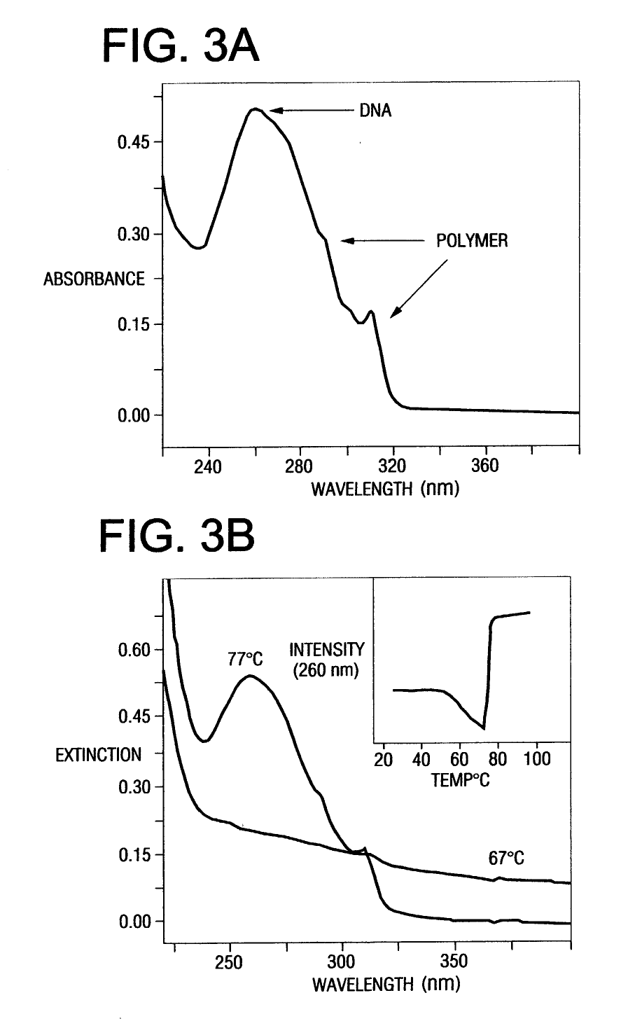

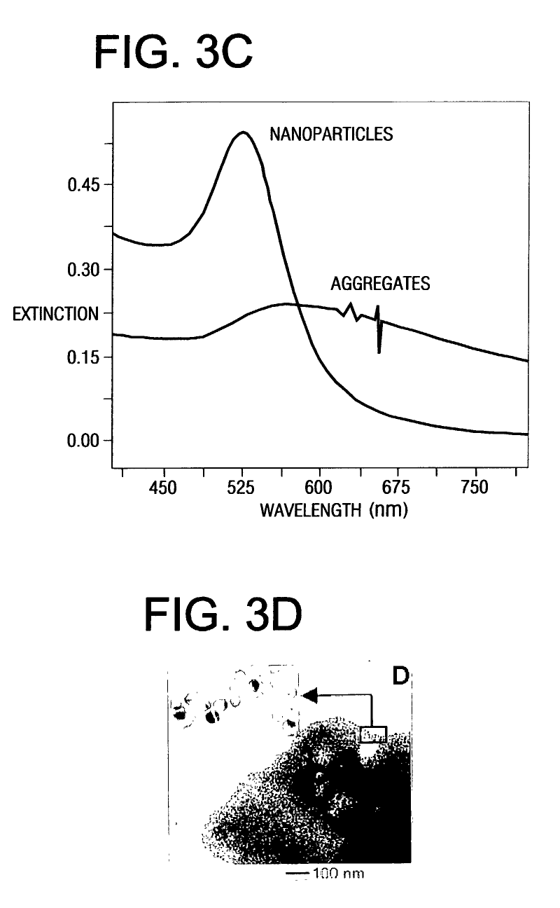

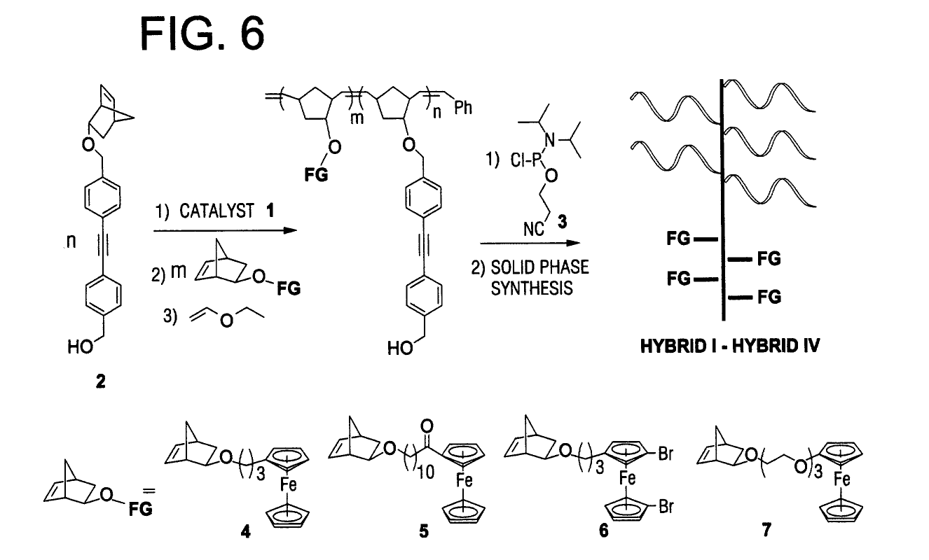

ROMP can provide polymers of varying average lengths (i.e. varying degree of polymerization, DP) depending on the ratio of monomer to ROMP catalyst (i.e., initiator). The polymer (or polymer template) is preferably prepared by polymerizing one or more monomers using a metal carbene catalyst (i.e., a compound containing a metal carbene (M=CR⁴R⁵) bond that catalyzes metathesis reactions, wherein the R groups are each independently H or an organic group, and “M” represents a metal (preferably, ruthenium or osmium) bonded to one or more ligands in a ligand sphere). Specific examples of suitable catalysts include, but are not limited to, Grubb's ruthenium metal carbene catalyst (Compound 41, FIG. 6) and the compounds shown in FIG. 3 and disclosed in Kingsbury et al., J. Amer. Chem. Soc., 121, 791-799 (1999); Schwab et al., J. Amer. Chem. Soc., 118, 100-110 (1996); Dias et al., Organometallics, 17, 2758-2767 (1998); del Rio et al., Tetrahedron Lett., 40, 1401-1404 (1999); Furstner et al., Chem. Commun., 95-96 (1999); Weskamp et al., Angew. Chem., Int. Ed. Engl., 37, 2490-2493 (1998); and Scholl et al., Tetrahedron Lett., 40, 2247-2250 (1999). Others include those disclosed in, for example, U.S. Pat. No. 5,831,108 (Grubbs et al.), U.S. Pat. No. 5,342,909 (Grubbs et al.), U.S. Pat. No. 5,710,298 (Grubbs et al.), U.S. Pat. No. 5,312,940 (Grubbs et al.), U.S. Pat. No. 5,750,815 (Grubbs et al.), U.S. Pat. No. 5,880,231 (Grubbs et al.), U.S. Pat. No. 5,849,851 (Grubbs et al.), and U.S. Pat. No. 4,883,851 (Grubbs et al.). Generally, suitable catalysts are ruthenium and osilum carbene complex catalysts disclosed in the above cited references.

The preferred ruthenium and osmium carbene complex catalysts include those which are stable in the presence of a variety of functional groups including hycdroxyl, thiol, thioetlher, ketone, aldehyde, ester, ether, amine, imine, amide, nitro, carboxylic acid, disulfide, carbonate, isocyanate, carbodiimide, carboalkoxy, peroxy, anhydride, carbamate, and halogen. When the catalysts are stable in the presence of these groups, the starting monomers, impurities in the monomer, any substituent groups on the catalyst, and other additives may include one or more of the above listed groups without deactivating the catalysts.

The catalyst preferably includes a ruthenium or osmium metal center that is in a +2 oxidation state, has an electron count of 16, and is pentacoordinated. These ruthenium or osmium carbene complex catalysts may be represented by the formula:

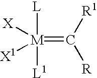

where:

M is O or Ru;

R and R¹ may be the same or different and may be hydrogen or a substituent group which may be C₂-C₂₀ alkenyl, C₂-C₂₀ alkynyl, C₁-C₂₀ alkyl, aryl, C₁-C₂₀ carboxylate, C₁-C₂₀ alkoxy, C₂-C₂₀ alkenyloxy, C₂-C₂₀ alkynyloxy, aryloxy, C₂-C₂₀ alkoxycarbonyl, C₁-C₂₀ alkylthio, C₁-C₂₀ alkylsulfonyl and C₁-C₂₀ alkylsulfiniyl. Optionally, the substituent group may be substituted with one or more groups selected from C₁-C₅ alkyl, halide, C₁-C₅ alkoxy, and phuenyl. The phenyl group may optionally be substituted with one or more groups selected from halide, C₁-C₅ alkyl, and C₁-C₅ alkoxy. Optionally, the substituent group may be substituted with one or more functional groups selected from hydroxyl, thiol, thioethler, ketone, aldehyde, ester, ether, amine, imine, amide, nitro, carboxylic acid, disulfide carbonate, isocyanate, carbodiimide, carboal koxy, peroxy, anhydride, carbamate, and halogen.

In a preferred embodiment, R and R¹ are the same or different and may be hydrogen, substituted aryl, unsubstituted aryl, substituted vinyl, and unsubstituted vinyl; where the substituted aryl and substituted vinyl are each substituted with one or more groups selected from hydroxyl, thiol, thioether, ketone, aldehyde, ester, ether, amine, imine, amide, nitro, carboxylic acid, disulfide, carbonate, isocyanate, carbodiimide, carboalkoxy, peroxy, anhydride, carbamate, and halogen, C₁-C₅ allcyl, C₁-C₅ alkoxy, unsubstituted phenyl, and phenyl substituted with halide, C₁-C₅ alkyl or C₁-C₅ alkoxy;

X and X¹ may be the same or different and may generally be hydrogen or any anionic liganid. An anionic ligand is any ligand which when removed from a metal center in its closed shell electron configuration has a negative charge. In a preferred embodiment, X and X¹ are thre same or different and may be halogen, hydrogen or a substituent group selected from C₁-C₂₀ alkyl, aryl, C₁-C₂₀ alkoxide, aryloxide, C₁-C₂₀ alkyldiketone, aryldiketonate, C₁-C₂₀ carboxylate, aryl or C₁-C₂₀ allcylsulfonate, C₁-C₂₀ alkylthio, C₁-C₂₀ alkylsulfonyl, and C₁-C₂₀ alkylsulfinyl. The substituent groups may optionally be substituted with C₁-C₅ alkyl, halogen, C₁-C₅ akloxy or phenyl. The phenyl may be optionally substituted with halogen, C₁-C₅ alkyl, or C₁-C₅ alkoxy.

In a more preferred embodiment, X and X¹ are the same or different and may be Cl, Br, I, H or a substituent group selected from benzoate, C₁-C₅ carboxylate, C₁-C₅ alkyl, phenoxy, C₁-C₅ alkoxy, C₁-C₅ alkylthio, aryl, and C₁-C₅ alkyl sulfonate. The substituent groups may be optionally substituted with C₁-C₅ alkyl or a phenyl group. The phenyl group may optionally be substituted with halogen, C₁-C₅ alkyl or C₁-C₅ alkcoxy. In an even more preferred embodiment, X and X¹ are the same or different and are selected from C₁, CF₃ CO₂, CH₃ CO₂, CFH₂ CO₂, (CH₃)₃ CO, (CF₃)₂ (CH₃)CO, (CF₃) (CH₃)₂ CO, PhO, MeO, EtO, tosylate, mesylate, and trifluoromethanesulfonate.

In the most preferred embodiment, X and X¹ are both Cl; and L and L¹ may be the same or different and may be generally be any neutral electron donor. A neutral electron donor is any ligand which, when removed from a metal center in its closed shell electron configuration, has a neutral charge. In a preferred embodiment, L and L¹ may be the same or different and may be phosphines, sulfonated phospines, phosphites, phiosphinites, phosphonites, arsines, stibines, ethers, amines, amides, sulfoxides, carboxyls, nitrosyls, pyridines, and thioethers. In a more preferred embodiment, L and L¹ are the same or different and are phosphines of the formula PR³, R⁴ R⁵ where R³ is a secondary alkyl or cycloaklyl and R⁴ and R⁵ are the same or different and are aryl, C₁-C₁₀ primary alkyl, secondary alkyl, or cycloalkyl.

In the most preferred embodiment, L and L¹ are the same or different and are —P(cyclohexyl)₃, —P(cyclopentyl)₃, or —P(isopropyl)₃. L and L¹ may also be —P(phenyl)₃.

A preferred group of catalysts are those where M is Ru; R¹ and R are independently hydrogen or substituted or unsubstituted aryl or substituted or unsubstituted vinyl; X and X¹ are Cl; and L and L¹ are triphenylphosphines or trialkylphosphines such as tricyclopentylphosphine, tricyclohexylphosphine, and triisopropylphosphine. The substituted aryl and substituted vinyl. may each be substituted with one or more groups including C₁-C₅ alkyl, halide, C₁-C₅ alkoxy, and a phenyl group which may be optionally substituted with one or more halide, C₁-C₅ alkyl, or C₁-C₅ alkoxy groups. The substituted aryl and substituted vinyl may also be substituted with one or more functional groups including hydroxyl, thiol, thioether, ketone, aldehyde, ester, ether, amine, imine, amide, nitro, carboxylic acid, disulfide, carbonate, isocyanate, carbodiimide, carboalkoxy, peroxy, anhydride, carbamate, and halogen.

Particularly preferred catalysts can be represented by the formulas:

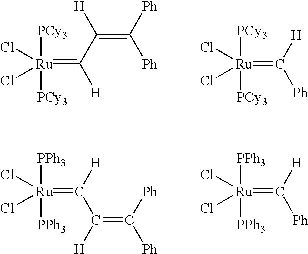

where Cy is cyclopentyl or cyclohexyl, and Ph is phenyl.

The most preferred catalysts can be represented by the formula:

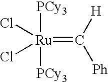

where Cy is cyclopenotyl or cyclohexyl, and Ph is phenyl.

The catalysts described above are useful in polymerization of a wide variety of olefin monomers through metathesis polymerization, particularly ROMP of cycloolefins.

Suitable monomers include olefins that can be polymerized by any of the ruthenium or osmium metathesis polymerization catalysts that were discussed arbove. Suitable monomers for use in the present invention have at least one polymerizable group (and often only one polymerizable group) and at least one functional group (used for subsequent modification for coupling to an oligonucleotide) and/or reporter label and result in a polymer or polymer template that is stable to the ROMP polymerization conditions. The olefin monomers may be unfunctionalized or functionalized to contain one or more functional groups selected from the group consisting of hydroxyl, thiol, thioether, ketone, aldehyde, ester, ether, amine, imine, amidie, nitro, carboxylic acid, disulfide, carbonate, isocyanate, carbodiimide, carboalkoxy, peroxy, anhydride, carbamate, and halogen.

The olefin may be a strained cyclic olefin, or unstrained cyclic olefin, each of which may be functionalized or unfunctionalized. Preferred monomers include functionalized or unfunctionalized cyclic olefins that are polymerized through ROMP reactions. This polymerization process includes contacting a functionalized or unfunctionalized cyclic olefin with a ruthenium or osmium metathesis catalysts discussed above. The cyclic olefins may be strained or unstrained and may be monocyclic, bicyclic, or multicyclic olefins. If the cyclic olefin is functionalized, it may contain one or more functional groups including hydroxyl, thiol, thioether, ketone, aldehyde, ester, ether, amine, imine, amide, nitro, carboxylic acid, disulfide, carbonate, isocyanate, carbodiimide, carboalkoxy, peroxy, anhydride, carbamate, and halogen.

Suitable cyclic olefin monomers include monomers disclosed in U.S. Pat. No. 4,943,621 to Janda, et al., U.S. Pat. No. 4,324,717 to Layer, and U.S. Pat. No. 4,301,306 to Layer, all of which are herein incorporated by reference.

Suitable cyclic olefin monomers include norbornene-type monomers which are characterized by the presence of at least one norbornene group which can be substituted or unsubstituted. Suitable norbornene type monomers include substituted norbornenes and unsubstituted norbornene, dicyclopentadiene, di(methyl) dicyclopentadiene, dilhydrodicyclopentadiene, cyclopentadiene trimers, tetramers of cyclopentadiene, tetracyclododecene, and substituted tetracyclododecenes.

Common norbornene-type monomers can be represented by the following formulas:

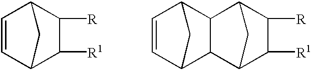

wherein R and R¹ may be the same or different and may be hydrogen or a substitute group which may be a halogen, C₁-C₁₂ alkyl groups, C₂-C₁₂ alkylene groups, C₆-C₁₂ cycloalkyl groups, C₆-C₁₂ cycloalkylene groups, and C₁-C₁₂ aryl groups or R and R¹ together form saturated or unsaturated cyclic groups of from 4 to 12 carbon atoms with the two ring carbon atoms connected thereto, said ring carbon atoms forming part of and contributing to the 4 to 12 carbon atoms in the cyclic group.

Less common norbornene type monomers of the following formulas are also suitable:

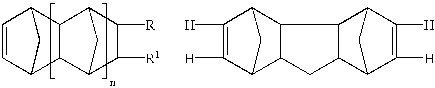

wherein R and R¹ have the same meaning as indicated above and n is greater than 1. For example, cyclopentadiene tetramers (n=2), cyclopentadiene pentamers (n=3) and hexacyclopentadecene (n=2) are suitable monomers for use in this invention.

Other specific examples of monomers suitable for use in this invention include: ethylidenenorbornene, methyltetracyclododecene, methylnorborinene, ethylnorbornene, dimethylnorbornene and similar derivatives, norbornadiene, cyclopentene, cycloheptene, cyclooctene, 7-oxanorbornene, 7-oxanorbomiene derivatives, 7-oxabicyclo[2.2.1]hept-5ene derivatives, 7-oxanorbornadiene, cyclododecene, 2-norbornene, also named bicyclo[2.2.1]-2-heptene and substituted bicyclic norbornenes, 5-methyl-2-norbornene, 5,6-dimethyl-2-norbornene, 5-ethyl-2-norbornene, 5-butyl-2-norbornene, 5-hexyl-2-norbornene, 5-dodecyl-2-norbornene, 5-isobutyl-2-norbornene, 5-octadecyl-2-norbornene, 5-isopropyl-2-norbornene, 5-phenyl-2-norbornene, 5-p-tolyl-2-norbornene, 5-a-naphthyl-2-norbornene, 5-cyclohexyl-2-norbornene, 5,5-dimethyl-2-norbornene, dicyclopentadiene (or cyclopentadiene dimer), dihydrodicyclopentadiene (orcyclopentene cyclopentadiene codimer), methyl-cyclopentadiene dimer, ethyl-cyclopentadiene dimer, tetracyclododecene, also named 1,2,3,4,4a,5,8,8a-octahydro-1,4:5,8-dimethyanonaphthalene 9-methyl-tetracyclo[6.2.1.1^{3,6.}0^{2,7}]-4-dodecene, also named 1,2,3,4,4a,5,8,8a-octahydro-2-metlhyl-4,4:5,8-dimethanonaphthalene 9-ethyl-tetracyclo [6.2.1.1^{3,6}0^{2,7}]-4-dodecene, 9-propyl-tetracyclo[6.2.1.1^{3,6}0^{2,7}]-4-dodecene, 9-hexyl-tetracyclo[6.2.1.1^{3,6}0^{2,7}]-4-dodecene, 9-decyl-tetracyclo[6.2.1.1^{3,6}0^{2,7}]-4-dodecene, 9,10-dimethyl-tetracyclo[6.2.1.1^{3,6}0^{2,7}]-4-dodecene, 9-ethlyl, 10-methyl-tetracyclo[6.2. 1.1^{3,6}0^{2,7}]-4-dodecene, 9-cyclohexyltetracyclo[6.2.1.1^{3,6}0^{2,7}]-4-dodecene, 9-chloro-tetracyclo[6.2.1.1^{3,6}0^{2,7}]-4-dodecene, 9-bromo-tetracyclo[6.2.1.1^{3,6}0^{2,7}]-4-dodecene, cyclopentadiene-trimer, methyl-cyclopentadiene-trimer, and the like.

In a preferred embodiment, the cyclic olefin is cyclobutene, dimethyl dicyclopentadiene, cyclopentene, cycloheptene, cyclooctene, cyclononene, cyclodecene, cyclooctadiene, cyclononadiene, cyclododecene, norbornene, norbornadiene, 7-oxanorbornene, 7-oxanorbornadiene, and dicyclopentadiene; each of which may be functionalized or unfunctionalized.

In a most preferred embodiment, the cyclic olefin is subsitututed norbornenes. The norbornenes include a functional group and/or reporter label that is attached to the norbornene via a linker. Any suitable linker of any suitable length may be used, including without limitation, linear or branched C₁-C₂₀ alkyls, C₁-C₂₀ alkyl ethers, aryl C₁-C₂₀ alkyls, aryl C₁-C₂₀ alkyls ethers, C₁-C₂₀ alkenyls, C₁-C₂₀ alkynyls, aryl C₁-C₂₀ alkynyls which may optionally substituted. A suitable reporter label may be attached including without limitation UV labels, fluorescent labels, radiolabels, redox labels, et.c.

A preferred monomer is a norbornenyl-substituted ferrocene. Preferrably, the monomer is a norbornene bound to a ferrocene via a linker. A preferred norbornenyl-substituted ferrocene (NSF) monomer has the following formula:

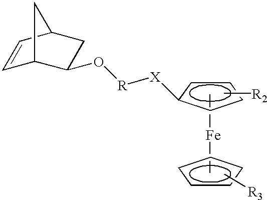

Wherein the linker R may be any suitable moiety for connecting the ferrocene or any other electrochemical tag to the norbornene structure. Representative examples of R include C₁-C₂₀ alkyl, C₁-C₂₀ alkynyl, C₁-C₂₀ alkenyl, aryl C₁-C₂₀ alkyl, C₁-C₂₀ alkoxy, aryl C₁-C₂₀ alkynyl, aryl C₁-C₂₀ alkenyl, aryl C₁-C₂₀ alkoxy, —(CH₂CH₂O)ₙ— wherein n=1-20. R₂ and R₃ may be independently H, halogen, —OH, or C₁-C₂₀ alkyl or alkoxide.

The invention contemplates the preparation of ROMP homopolymers, as well as random and block co-polymers, terpolymers, random copolymers with more than three different monomers, and multiblock copolymers of the suitable monomers discussed above. Using NSF monomers, oligonucleotide-modified ROMP block co-polymers may be designed with highly tailorable redox-activities. Based on the block co-polymer strategy described in the Examples below, one can incorporate about four different NSF monomers as indicators. To increase the number of indicators and thus use their redox potentials as a type of barcode to serve as an indicator for the presence of a target nucleic acid or oligonucleotide, multiblock, e.g., triblock, co-polymers containing different NSF derivatives can be used. By adjusting the ratio between the redox active blocks during the ROMP reaction, one can generate many many different indicators rather than four in the case of diblock co-polymers which is useful for multichannel DNA detection.

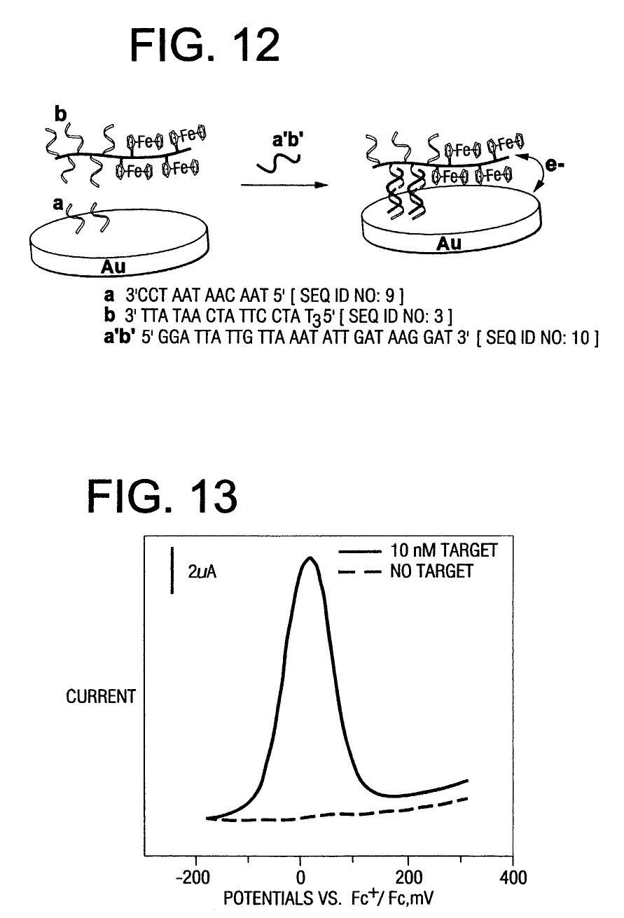

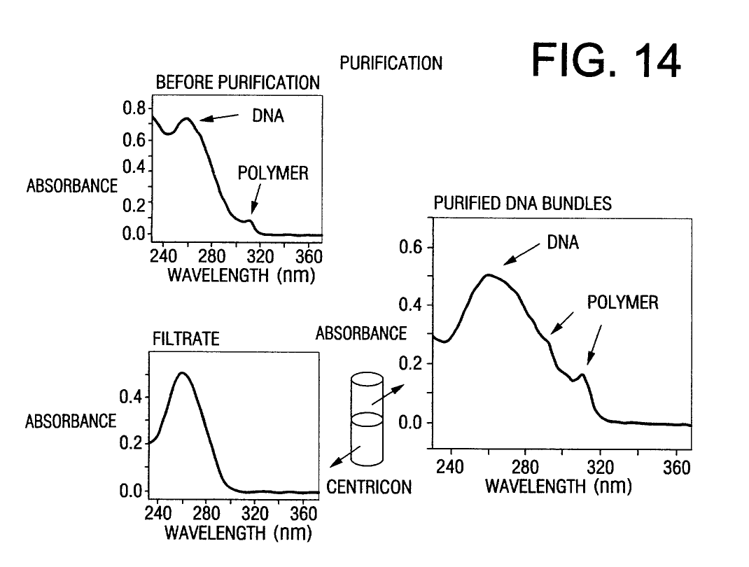

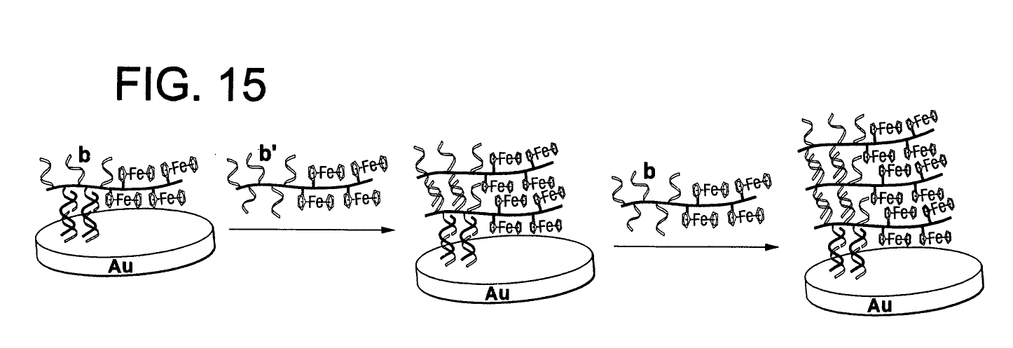

In polymers prepared with monomers having functional groups for subsequent modification, these polymers may be used as templates for a post-polymerization reaction with a chlorophosphoramidite reagent under suitable conditions to produce a modified template suitable for coupling to DNA using standard DNA solid phase synthetic techniques. Any chlorophosphoramidite reagent and any suitable modification conditions may be used to prepare the chlorophosphoramidite modified polymer template. Suitable, but non-limiting examples of chlrophosphoramidite reagents include 2-cyanoethyl diisopropylchlorophosphoramidite or 2-cyanoethyl tetraisopropylchlorophosphoramidite. Using this reaction, oligonucleotides are readily attached to the polymer backbone to produced novel oligonucleotide-modified ROMP polymers or co-polymers have a well-defined polymer structure. As shown in the Examples below, three dimensional aggregated structures comprised of ROMP polymers or co-polymers having complementary oligonucleotides can be produced and have extended hybridization networks which precipate reversibly from aqueous solution. This establishes that the attachment of the oligonucleotides to the polymers and existence of one or more blocks in the co-polymers do not interfere with the recognition properties of the DNA. One can exploit the DNA recognition properties of the oligonucleotide-modified polymers or co-polymers for detection of target nucleic acids or other oligonucleotides and the further preparation of new materials. For instance, monomers such as norbornene linked to electrochemically active molecules can be used for preparing oligonucleotide-modified ROMP block copolymers with electrochemical tags and having redox activity that can be used for the electrochemical detection of target nucleic acids or other oligonucleotides using cyclic voltammetry or pulse voltammetry. See for instance FIG. 12. For signal amplification, a complementary oligonucleotide-modified ROMP block copolymer may be used to bind to any unbound oligonucleotide bound to the ROMP block copolymer involved in the initial complexation with a target nucleic acid and oligonucleotides bound to the gold electrode surface. See FIG. 14.

DNA hybridization interactions between oligonucleotide-modified ROMP polymers or copolymers with complementary oligonucleotides labeled particles may be exploited to prepare materials with new properties. Examples of particles including, without limitation, latex particles, polystrene particles, and particles such as metallic particles (e.g., gold), semiconductor particles (e.g., CdSe/ZnS core/shell), insulator particles, polymer particles (e.g., polyacrylates), inorganic particles (e.g., silica or metal oxide) or combinations there of. Numerous examples of suitable particles and methods for preparation are described, for instance, in Mirkin U.S. Pat. No. 6,361,944; PCT/US01/01190; PCT/US01/10071; and U.S. Ser. No. 09/603,830, file Jun. 26, 2000; and Ser. No. 10/008,979, filed Dec. 7, 2001. The present invention contemplates the use of any suitable particle having oligonucleotides attached thereto that are suitable for use in detection assays. In practicing this invention, however, nanoparticles are preferred. The size, shape and chemical composition of the particles will contribute to the properties of the resulting probe including the DNA barcode. These properties include optical properties, optoelectronic properties, electrochemical properties, electronic properties, stability in various solutions, pore and channel size variation, ability to separate bioactive molecules while acting as a filter, etc. The use of mixtures of particles having different sizes, shapes and/or chemical compositions, as well as the use of particles having uniform sizes, shapes and chemical composition, are contemplated. Examples of suitable particles include, without limitation, nano- and microsized core particles, aggregated particles, isotropic (such as spherical particles) and anisotropic particles (such as non-spherical rods, tetrahedral, prisms) and core-shell particles such as the ones described in U.S. Patent application Ser. No. 10/034,451, filed Dec. 28, 2002 and International application no. PCT/US01/50825, filed Dec. 28, 2002, which are incorporated by reference in their entirety.

Particles useful in the practice of the invention include metal (e.g., gold, silver, copper and platinum), semiconductor (e.g., CdSe, CdS, and CdS or CdSe coated with ZnS) and magnetic (e.g., ferromagnetite) colloidal materials. Other particles useful in the practice of the invention include ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs. The size of the particles is preferably from about 5 nm to about 150 nm (mean diameter), more preferably from about 5 to about 50 nm, most preferably from about 10 to about 30 nm. The particles may also be rods, prisms, or tetrahedra.

Methods of making metal, semiconductor and magnetic particles are well-known in the art. See, e.g., Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Taransactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988).

Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs particles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky et al., J. Am. Chem. Soc., 112, 9438 (1990); Ushida et al., J. Phys. Chem., 95, 5382 (1992).

Suitable particles are also commercially available from, e.g. Ted Pella, Inc. (gold), Amersham Corporation (gold) and Nanoprobes, Inc. (gold).

Presently preferred for use in detecting nucleic acids are gold particles. Gold colloidal particles have high extinction coefficients for the bands that give rise to their beautiful colors. These intense colors change with particle size, concentration, interparticle distance, and extent of aggregation and shape (geometry) of the aggregates, making these materials particularly attractive for colorimetric assays. For instance, hybridization of oligonucleotides attached to gold particles with oligonucleotides and nucleic acids results in an immediate color change visible to the naked eye (see, e.g., the Examples).

In an attempt to incorporate DNA into ROMP polymers, post-polymerization modification of preformed polymers with DNA was performed. For this task, the norbornenyl-modified alcohol 2, which is characterized by a diphenylacetylene spacer which separates the alcohol from the polymerizable norbornene, proved to be extremely useful. Starting from 5-exo-norbornen-2-ol, 2 is isolated in five high-yielding steps. With its strong absorption maximum at 304 nm (Ext. coef.=26000), the diphenylaceylene component serves as a convenient UV-tag that can be used to monitor reactivity.

The synthesis of poly2 and reaction of this polymer with the chlorophosphoramidite 3 resulted in a material with a single resonance in the ³¹P NMR spectra at 149.2 ppm. This result is consistent with that observed for the monomeric analogue. Subsequent coupling to CPG-supported DNA using the syringe technique, followed by deprotection of the DNA from the solid support in aqueous ammonia at 60° C., yielded the desired hybrid product poly2. The DNA is connected to the polymer at the 5′-end. Purification of the hybrid product from failure strands was achieved using ultrafiltration.

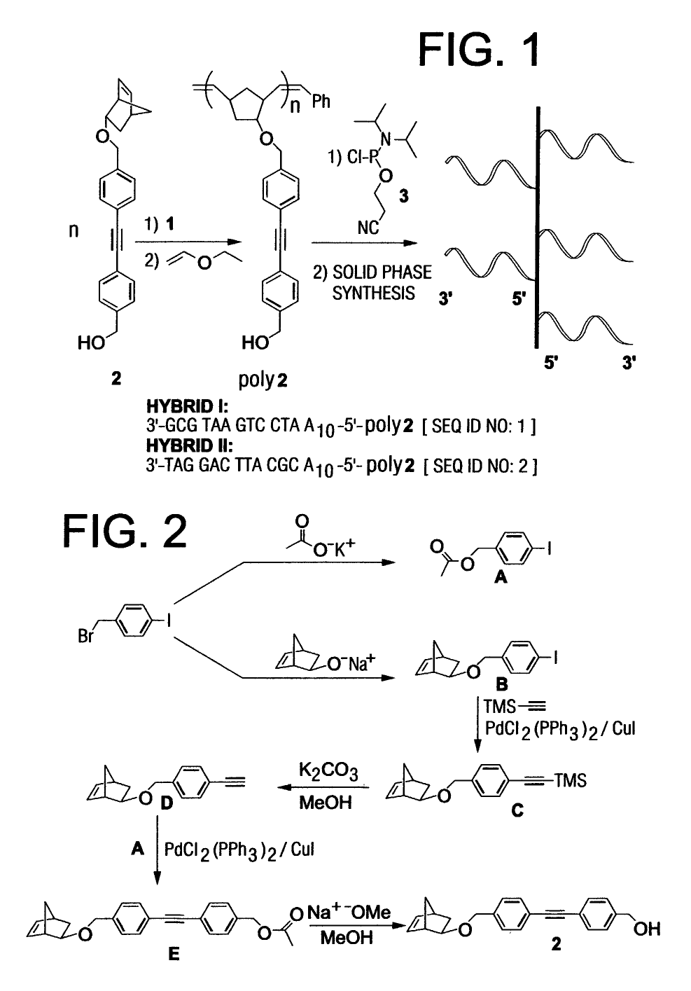

As a first demonstration, this general experimental strategy was used to isolate two polymers modified with complementary 12-mers of DNA with A₁₀ spacers (FIG. 1, Hybrid-I and Hybrid-II). The UV-spectra of the purified DNA/polymer hybrids in water provides strong evidence that the DNA is attached to the polymer backbone (FIG. 3A). The UV-Vis absorption maximum at 310 nm demonstrates that the diphenylacetylene backbone is present, which suggests that the water-soluble oligonucleotides are covalently linked to the hydrophobic polymer structure. Using experimental and calculated extinction coefficients for the polymer and the oligonucleotides, and assuming a repeat unit of the polymer consistent with the stoichiometry of its synthesis, we estimate that there are, on average, 5 DNA stands attached to each polymer chain. This translates to 30% occupation of the potential polymer attachment sites by DNA strands.

When solutions of Hybrid-I and Hybrid-II are mixed in a PBS buffer solution (PBS=0.3 M NaCl, 10 mM phosphate, pH 7), hybridization triggers the formation of an extended network aggregate of linked polymers, which is signaled by the immediate formation of a white precipitate. Presumably, this occurs because each polymer is modified with more than one strand of DNA, which leads to cooperative binding between many DNA functionalized complementary polymer stands, as evidenced by the sharp melting transition (inset of FIG. 3B). As expected, this hybridzation process is thermally reversible. These studies demonstrate that attachment to the polymer does not hinder the recognition properties of the oligonucleotides.

The DNA/polymer conjugate was utilized to form particle assemblies. When a PBS buffer solution of Hybrid-I (12 μL of 8.3 μM in DNA) is mixed with a PBS buffer solution of 13 nm Au particles (260 μL of 9.7 nM in particle) modified with complementary DNA strands,² the formation of three-dimentional particle aggregates is signaled by the diagnostic shift in the surface plasmon resonance of the particles (from 520 nm to 570 nm, FIG. 3C) and a corresponding change in color (from red to purple).^{2,3} Transmission electron microscopy studies reveal a high networked aggregate (FIG. 3D). Control experiments in which a solution of the same particles was mixed with a buffered solution of Hybrid-II (which is non-complementary) resulted in no aggregate formation under nearly identical conditions.

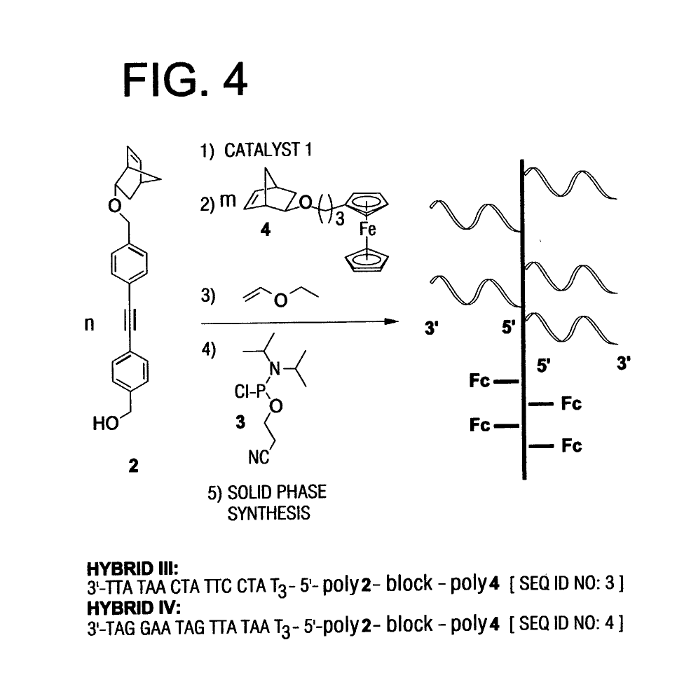

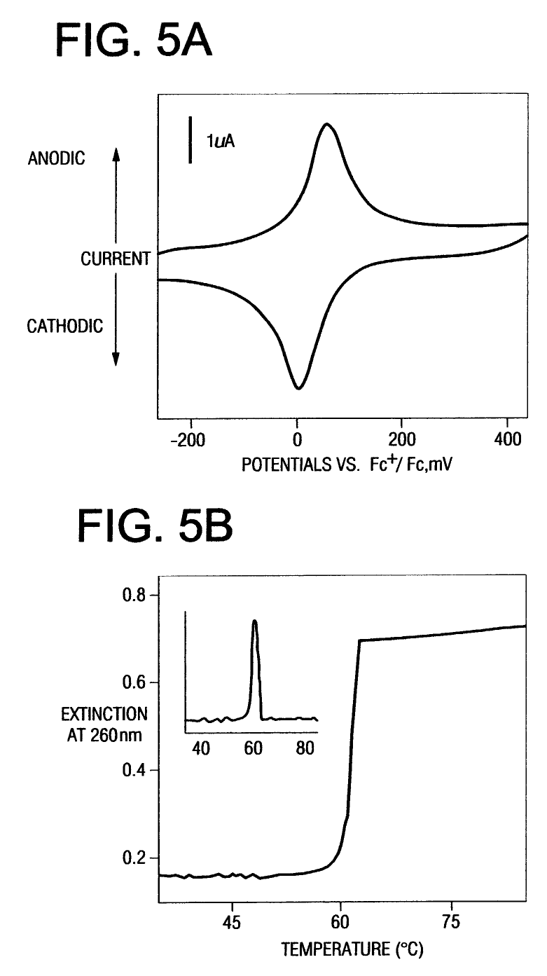

Given the exceptional functional group tolerance of catalyst 1 in ROMP, it has been hypothesized that this chemistry could be extended to block copolymers of 2 with a number of norbornene monomers, thereby imparting tailorable functionality to branched DNA structures. The strategy for the synthesis of block copolymer branched DNA is outlined in FIG. 4. In these experiments, monomer 2 is mixed with a catalytic amount of 1 in dry THF. After 1 h, the polymerization of the first block was determined to be complete by ¹H NMR spectroscopy. Subsequent addition and polymerization of a second norbornene monomer, followed by the injection of ethyl vinyl ether to terminate the reaction, yielded block copolymers with the desired structure. When the norbornenyl-modified ferrocene 4 was used as a second block, post-polymerization modification with 3 and solid phase DNA synthesis led to the isolation of Hybrid III and Hybrid IV. Electrochemical measurements confirm the presence of the ferrocenes (Fc) in these hybrids (E½=33 mV vs Fc/Fc⁺, FIG. 5A). When PBS-buffered solutions of these hybrids were mixed, thermally reversible aggregate formation was again observed. Also, a sharp melting transition was observed, consistent with our proposed structure (FIG. 5B).

The aforementioned data illustrates that post-polymerization modification of ROMP polymers and block copolymers with DNA can lead to DNA/polymer hybrid materials with a number of interesting properties associated with the hybrid structure. The experiments described herein reveal that the recognition properties of the DNA strands are not adversely affected by attachment to the polymer. These new structures can be prepared with properties and function that depend upon the choice of ROMP monomer and DNA branch sites. Since the synthesis of block copolymers of 2 with other norbornenyl-modified compounds is a facile process, the isolation of other novel and potentially useful macromolecular hybrid materials should be readily accomplished by utilizing variations of the strategy presented herein. Details of the synthesis and characterization of 2, 4, poly2, poly2-block-poly4 and other experimental procedures are described below.

It is to be noted that the term “a” or “an” entity refers to one or more of that entity. For example, “a characteristic” refers to one or more characteristics or at least one characteristic. As such, the terms “a” (or “an”), “one or more” and “at least one” are used interchangeably herein. It is also to be noted that the terms “comprising”, “including”, and “having” have been used interchangeably.

Objects and advantages of this invention are further illustrated by the following examples, but the particular materials and amounts thereof recited in these examples, as well as other conditions and details, should not be construed to unduly limit this invention.

### EXAMPLES

General Considerations. All reactions were carried out under a dry nitrogen atmosphere using either standard Schlenk techniques or in an inert-atmosphere glovebox unless otherwise noted. All solvents were distilled under nitrogen and saturated with nitrogen prior to use. ¹H and ¹³C NMR spectra were recorded on either a Varian Mercury 300 FT-NMR spectrometer (300 MHz for ¹H NMR, 125 MHz for ¹³C NMR) or a Varian Mercury 400 FT-NMR spectrometer (400 MHz for ¹H, 100 MHz for ¹³C. ¹H NMR data are reported as follows: chemical shift (multiplicity (b=broad singlet, s=singlet, d=doublet, t=triplet, q=quartet, and m=multiplet), integration, and peak assignments). ¹H and ¹³C chemical shifts are reported in ppm downfield from tetramethylsilane (TMS, δ scale) with the residual solvent resonances as internal standards, while peak assignments were made with the aid of ACD Laboratories software. High-resolution electron-impact mass spectrometry (HREIMS) data was obtained on a VG 70-SE instrument. High-performance liquid chromatography was performed using a HP series 1100 HPLC.

Gel permeation chromatography (GPC) was performed on either a Waters GPC system equipped with a 515 HPLC pump, a 486 Tunable Absorbance Detector, and two serially connected Supelco Progel-TSK GM H6 columns (Column dimensions 30 cm×7.5 mm×⅜ in (length×I.D.×.O.D.)). THF was used as the eluent at a flow rate of 1 mL/min and the instrument was califbrated with six polystyrene standards (Mₙ=2,430 to 212,400 Daltons) from Aldrich). Alternatively, GPC was also carried out on a Waters Breeze system equipped with a 1525 HPLC pump, a 2487 Dual Wavelength Absorbance Detector, a 2410 Refractive Index Dectector, and a Shodex GPC Mixed-Bed KF-806-L column connected in series with a Shodix GPC Mixed-Bed KF-803-L (column dimensions 300 mm×8 mm for both). THF was used as the eluent at a flow rate of 1 mL/min and the instrument was calibrated with an Aldrich kit containing seventeen polystyrene standards (Mn=760 to 1,880,000 Daltons). All flash column chromatography was carried out using a 56 mm inner-diameter column containing a 200 mm plug of silica gel under a positive pressure of nitrogen, unless otherwise noted. UV-vis spectra were recorded using a Hewlett Packard (HP) 8452A diode-array spectrophotometer. Transmission electron microscopy was performed on a Hitachi 8100 microscope. A typical sample was prepared by dropping 10 uL of aggregate solution onto a holey carbon TEM grid, followed by wicking the solution away. The grid was subsequently dried and imaged. Electronic absorption spectra were recorded using a Hewlett Packard (HP) 8452A diode array spectrophotometer. Melting analyses were performed using an HP 8453 diode-array spectrophotometer equipped with a HP 89090A Peltier temperature controller. Electrochemical measurements were carried out in a three-electrode cell using a BAS 100B (Bioanalytical Systems Inc). Au-thin film working electrodes were prepared on silicon wafers by thermal evaporation immediately prior to use. For cyclic voltametry measurements, films of hybrids were prepared by spreading a PBS solution of the copolymer on the electrode and allowing the solvent to evaporate at room temperature. The reference electrode was a silver wire, while a platinum wire was used as the counter electrode. The supporting electrolyte was 0.2 M [(n-Bu)₄N]PF₆ in CH₂Cl₂, and all experiments were carried out at room temperature after the solution was degassed by purging with nitrogen for 10 min. Alternating Current (AC) voltammograms were acquired in a low frequency (10 Hz) mode at a peak AC voltage amplitude of 25 mV.

Materials. The catalyst Cl₂Ru(PCy₃)₂═CHPh¹⁸ (1) and 5-exo-norbornen-2-ol ^{19,20} were prepared from literature procedures. Alternatively, catalyst 1 can also be purchased from Strem Chemicals. The synthesis and purification of (alkanethiol)-modified oligonucleotides was performed as described elsewhere.²¹ Acetonitrile, CHCl₃, NEt₃, and CH₂Cl₂ were distilled over calcium hydride. Tetrahydrofuran (THF), toluene, and diethyl ether were distilled over sodium/benzophenone. Methanol was distilled over Mg(OMe)₂. All solvents were distilled under nitrogen and saturated with nitrogen prior to use. Deuterated solvents were purchased from Cambridge Isotope Laboratories and used without further purification, except for CDCl₃, which was distilled over calcium hydride and vacuum transferred into an air-tight solvent bulb prior to transfer into the inert-atmosphere glovebox. All other reagents were purchased from Aldrich or Lancaster Synthesis and used without further purification.

### Example 1

### Synthesis of a DNA-modified ROMP Polymer

This Example describes the preparation of a ROMP homopolymer from a norbornene monomer 2 possessing a UV tag followed by post-polymerization modification of the polymer to attach oligonucleotides using standard DNA solid phase synthetic techniques. Synthesis of 2. The synthesis of 2 was accomplished according to the procedure outlined in FIGS. 1 and 2. The syntheses and characterizations of all new compounds in FIG. 2 are outlined below:

Synthesis of 4-iodobenyl acetate (A). Potassium acetate (1.50 g, 15.3 mmol) and 4-iodobenzyl bromide (3.00 g, 10.1 mmol) were mixed with ethanol (40 mL) in a 100-mL Schlenk flask and refluxed overnight. Upon cooling to room temperature, the mixture was poured into water (200 mL) and extracted with ether (3×100 mL). The organic portions were collected, washed with water (200 mL), dried over sodium sulfate, and filtered into a 500-mL round bottom flask. The solvent was removed under vacuum. Purification on silica using 30% CH₂Cl₂ in hexanes as an eluent yielded the desired product A (2.59 g, 2.59 mmol, 93%) as a white solid. ¹H NMR (C₆D₆): δ 1.59 (s, 3H, —CH₃), 4.68 (s, 2H, —CH₂), 6.62 (m, 2H, aromatic-H), 7.38 (m, 2H, aromatic-H)). ¹³C NMR (CDCl₃): δ 21.15, 65.72, 94.12, 130.27, 135.73, 137.83, 170.91. HREIMS: Calcd for C₉H₉₁O₂: 275.9647. Found: 275.9647.

Synthesis of exo-5-norbornene-2-(4-iodobenzyloxy) (B). In an inert atmosphere glovebox, exo-5-norbornene-2-ol (1.00 g, 9.08 mmol) was weighed into a 50-mL Schlenk flask. THF (15 mL) was added, and the solution was stirred while oil-free sodium metal (300 mg, 13.0 mmol) was added. The mixture was then taken out of the glovebox, refluxed for 12 h under a nitrogen bubbler, and allowed to cool to room temperature. In a separate 100-mL Schlenk flask, 4-iodobenzyl bromide (1.70 g, 5.73 mmol) was dissolved in dry THF (15 mL) under nitrogen. The cooled solution of deprotonated exo-5-norbornen-2-ol was then transferred via cannula to the benzyl bromide solution with vigorous stirring. The flask was capped with a condenser and the mixture was refluxed for an additional 12 h under a nitrogen bubbler. Upon cooling to room temperature, the reaction mixture was poured into ether (50 mL) and washed successively with water (50 mL), 0.1 M NaOH (50 mL), 1.0 M HCl (50 mL), and brine (50 mL). The organic layer was collected, dried over sodium sulfate, and filtered into a 500-mL round bottom flask. The solvent was removed under vacuum. Column chromatography on silica gel with 30% CH₂Cl₂ in hexanes as the eluent gave the desired product B (1.75 g, 5.36 mmol, 94%) as a clear oil. ¹H NMR (C₆D₆): δ 1.35-1.82 (m, 4H, 3- and 7-norbornenyl-H)), 2.60 (b, 1H, 1-norbornenyl-H), 2.80 (b, 1H, 4-norbornenyl-H), 3.38 (m, 1H, 2-norbornenyl-H), 4.10 (m, 2H, CH₂—O), 5.76 (m, 1H, 6-norbornenyl-H), 6.03 (m, 1H, 5-norbornenyl-H), 6.79 (m, 2H, aromatic-H), 7.48 (m, 2H, aromatic-H). ¹³C NMR (CDCl₃): δ 34.68, 40.60, 46.22, 46.65, 70.67, 80.41, 92.96, 129.64, 133.27, 137.58, 138.82, 140.98. HREIMS: Calcd for C₁₄H₁₅IO: 326.0168. Found: 326.0168.

Synthesis of exo-5-norbornene-2-((4-(trimethylsilyl)acetylenyl)benzyloxy)(C). Into a 50-mL Schlenk flask was added B (2.00 g, 6.13 mmol), PdCl₂(PPh₃)₂ (200 mg,0.285 mmol, 4.6 mol %), and CuI (100 mg, 0.525 mmol. 8.6 mol %). The flask was placed in a dry box and charged with triethylamine (30 mL). To this stirring solution was added (trimethylsilyl)acetylene (1.50 mL, 10.6 mmol). The mixture was stirred for 12 h at 50° C., during which time a significant amount of solid formed. Upon cooling to room temperature, the mixture was poured into ether (50 mL) and filtered into a 250-mL round bottom flask. The solvent was removed under vacuum. Column chromatography on silica gel with 30% CH₂Cl₂ in hexanes as the eluent gave the desired product C (1.65 g, 5.56 mmol, 91%) as a clear oil. ¹H NMR (CDCl₃): δ 0.26 (s, 9H, —Si(CH₃)₃), 1.41-1.77 (m, 4H, 3- and 7-norbornenyl-H), 2.82 (b, 1H, 1-norbornenyl-H ), 2.93 (b, 1H, 4-norbornenyl-H), 3.58 (m, 1H, 2-norbornenyl-H), 4.53 (m, 2H, —CH₂—O), 5.92 (m, 1H, 6-norbornenyl-H), 6.20 (m, 1H, 5-norbornenyl-H), 7.28 (m, 2H, aromatic-H), 7.44 (m, 2H, aromatic-H). ¹³C NMR (CDCl₃): δ 0.21, 34.70, 40.62, 46.24, 46.67, 70.97, 80.43, 94.11, 105.28, 122.25, 127.42, 132.16, 133.31, 139.70, 140.97. HREIMS: Calcd for C₁₉H₂₄OSi: 296.1596. Found 296.1593.

Synthesis of exo-5-norbornene-2-((4-acetylenyl)benzyloxy) (D). Into a 100-mL Schlenk flask was added C (1.10 g, 4.05 mmol) and K₂CO₃ (15 mg). The flask was placed under nitrogen, charged with 30 mL of degassed CH₂Cl₂ and 50 mL of degassed MeOH, and covered with foil. The mixture was stirred at room temperature for 8 h, filtered into a round bottom flask, and the solvent was removed under vacuum. Column chromatography on silica gel with 30% CH₂Cl₂ in hexanes as the eluent gave the desired product D (807 mg, 3.60 mmol, 97%) as a clear oil. ¹H NMR (CDCl₃): δ 1.42-1.77 (m, 4H, 3- and 7-norbornenyl-H), 2.83 (b, 1H, 1-norbornenyl-H), 2.94 (b, 1H, 4-norbornenyl-H), 3.06 (s, 1H, C/CH), 3.59 (m, 1H, 2-norbornenyl-H), 4.54 (m, 2H, —CH₂—O), 5.92 (m, 1H, 6-norbornenyl-H), 6.20 (m, 1H, 5-norbornenyl-H), 7.31 (m, 2H, aromatic-H), 7.48 (m, 2H, aromatic-B). ¹³C NMR (CDCl₃): δ 34.69, 40.61, 46.23, 46.66, 70.89, 77.20, 80.47, 83.83, 121.19, 127.51, 132.32, 133.28, 140.05, 140.97. HREIMS: Calcd for C₁₆H6O: 224.1201. Found: 224.1201.

Synthesis of (a-(exo-5-norbornene-2-oxy)-α′-acetyl)ditolylacetylene (E). Into a 50-mL Schlenk flask was added D (550 mg, 2.45 mmol), A (65 mg, 2.45 mmol), PdCl₂(PPh₃)₂ (120 mg, 0.171 mmol, 7 mol %), and CuI (60 mg, 0.315 mmol, 12.9 mol %). The flask was placed in a dry box and charged with triethylamine (30 mL). The mixture was stirred for 12 h at 50° C., during which time a significant amount of solid formed. Upon cooling to room temperature, the mixture was poured into ether (50 mL) and filtered into a 250-mL round bottom flask. The solvent was removed under vacuum. Column chromatography on silica gel with 10% ethyl acetate in hexanes as the eluent gave the desired product E (880 mg, 2.36 mmol, 96%) as a white solid. ¹H NMR (CDCl₃): δ 1.47-1.79 (m, 4H, 3- and 7-norbornenyl-H), 2.13 (s, 3H, CH₃), 2.84 (b, 1H, 1-norbornenyl-H), 2.96 (b, 1H, 4-norbornenyl-H), 3.61 (m, 1H, 2-norbornenyl-H), 4.55 (m, 2H, —CH₂—O-norbornene), 5.12 (s, 2H, CH₂—O-acetate), 5.92 (m, 1H, 6-norbornenyl-H), 6.21 (m, 1H, 5-norbornenyl-H), 7.34 (m, 4H, aromatic-H), 7.54 (m, 4H, aromatic-H).

¹³C NMR (CDCl₃): δ 21.19, 34.69, 40.60, 46.23, 46.66, 66.04, 70.96, 80.45, 89.02, 90.03, 122.22, 123.43, 127.60, 128.30, 131.80, 131.92, 133.29, 136.11, 139.58, 140.96, 171.01. HREIMS: Calcdfor C₂₅H₂₄O₃: 372.1725. Found: 372.1725.

Synthesis of (α-(exo-5-norbornene-2-oxy)-α′-hydroxy)ditolylacetylene (2). Into a 50-mL Schlenk flask was added E (530 mg, 1.42 mmol). The flask was then evacuated and placed under nitrogen. To this solid was added a degassed solution of sodium methoxide (25 mL of a 2.8 mM solution, 0.071 mmol). The mixture was refluxed under nitrogen for 12 h, allowed to cool to room temperature, poured into water (200 mL), and extracted with ethyl acetate (3×100 mL). The organic portions were combined, washed with water (2×100 mL), dried over sodium sulfate, and filtered into a 500-mL round bottom flask. Removal of the solvent on a rotary evaporator, followed by extensive drying under vacuum at 50° C., yielded the desired product 2 (444 mg, 1.34 mmol, 94%) as a white solid. ¹H NMR (CDCl₃): δ 1.44-1.77 (m, 4H, 3- and 7-norbornenyl-H), 1.67 (s, 1H, —OH), 2.83 (b, 1H, 1-norbornenyl-H), 2.95 (b, 1H, 4-norbornenyl-H), 3.60 (m, 1H, 2-norbornenyl-H), 4.53 (m, 2H, —CH₂—O-norbornene), 4.71 (b, 2H, —CH₂—OH), 5.93 (m, 1H, 6-norbornenyl-H), 6.20 (m, 1H, 5-norbornenyl-H), 7.34 (m, 4H, aromatic-H), 7.52 (m, 4H, aromatic-h). ¹³C NMR (CDCl₃): δ 34.72, 40.65, 46.26, 46.72, 65.20, 71.03, 80.51, 89.27, 89.68, 122.40, 122.79, 127.03, 127.63, 131.81, 132.00, 133.33, 139.52, 140.99, 141.15. HREIMS: Calcd for C₂₃H₂₂O₂: 330.1620. Found: 330.1621. UV-Vis (MeOH): λₘₐₓ=304 nm (ε=26000), λₘₐₓ=286 nm (ε=28000); at 260 nm, ε=14000.

Synthesis of poly2. In an inert atmosphere glovebox, 2 (100 mg, 0.30 mmol) was weighed into a 25-mL round bottom flask equipped with a magnetic stir bar. Dry THF (4 mL) was added, followed by a solution of catalyst 1 (15.0 mg, 0.018 mmol) in dry THF (0.5 mL). The mixture was stirred for 120 min, after which time it was removed from the dry box and the polymerization was terminated with ethyl vinyl ether (1 mL). The polymer (poly2, 92 mg, 92%) was isolated by pouring the mixture into pentane, filtering, and repeatedly washing with fresh pentane (4×20 mL). GPC (THF): Mₙ=8,200; PDI=1.5.

Modification of poly2 with 3. Poly2 (50 mg) was dissolved in dry THF (5 mL). N,N-Diisopropylethylamine (200 μL) and 3 (50 mg, 0.21 mmol) were added and the mixture was stirred at room temperature for 2 h. The mixture was taken up in ethyl acetate (50 mL), washed with cold aqueous NaHCO₃ and water, dried over sodium sulfate, and concentrated to dryness to yield the desired product which was dissolved in CDCl₃ for ³¹P NMR analysis and then used directly in the next step. ³¹P NMR (CDCl₃): δ 149.2.

Attachment of DNA to Modified poly2. The attachment of DNA to modified poly2 was accomplished on CPG supports using conventional phosphoramidite chemistry²¹ and an automated DNA synthesizer (Expedite), except CDCl₃ was used as a solvent during the coupling step instead of acetonitrile, and purification was accomplished via ultrafiltration using a Centricon-50 instrument instead of HPLC (Figure Y).

Conclusion. In our attempts to incorporate DNA into ROMP polymers, we pursued post-polymerization modification of preformed polymers with DNA. For this task, the norbornenyl-modified alcohol 2, which is characterized by a diphenylacetylene spacer which separates the alcohol from the polymerizable norbornene, proved to be extremely useful (vide infra). Starting from 5-exo-norbornen-2-ol, 2 is isolated in five high-yielding steps. With its strong absorption maximum at 304 nm (Ext. coeff.=26000), the diphenylacetylene component serves as a convenient UV-tag which can be used to monitor reactivity (FIG. 3A).

The synthesis of poly2 and reaction of this polymer with the chlorophosphoramidite 3 resulted in a material with a single resonance (149.2 ppm) in the ³¹P NMR spectrum. This result is consistent with that observed for the monomeric analogue (i.e. the product of the coupling between 2 and 3). Subsequent coupling of poly2 to CPG-supported DNA using the syringe technique, followed by deprotection of the DNA from the solid support in aqueous ammonia at 60° C., yielded the desired hybrid product DNA-poly2 where the DNA is connected to the polymer at the 5′-end. Purification of the hybrid product from failure strands was achieved using ultrafiltration.

The general experimental strategy focuses on the isolation of two polymers modified with complementary 12-mers of DNA with A₁₀ spacers (FIG. 1, Hybrid I and Hybrid II). The UV-spectra of the purified DNA/polymer hybrids in water provides strong evidence that the DNA is attached to the polymer backbone (FIG. 3A). The absorption maximum at 310 nm demonstrates that the diphenylacetylene backbone is present, which suggests that the water-soluble oligonucleotides are covalently linked to the hydrophobic polymer structure. Using experimental and calculated extinction coefficients for the polymer and the oligonucleotides and assuming a DP of the polymer consistent with the stoichiometry of its synthesis, it is estimated that there is on average 5 DNA stands attached to each polymer chain. This translates to a 30% occupation of the potential DNA attachment sites.

When solutions of Hybrid-I and Hybrid-II are mixed in a PBS buffer solution (PBS=0.3 M NaCl, 10 mM phosphate, pH 7), hybridization triggers the formation of an extended network aggregate of linked polymers, which is signaled by the immediate formation of a white precipitate. Presumably, this occurs because each polymer is modified with more than one strand of DNA, which leads to cooperative binding between many DNA-functionalized complementary polymer stands, as evidenced by the sharp melting transition (inset of FIG. 3B). As expected, this hybridzation process is thermally reversible. These studies demonstrate that attachment to the polymer does not hinder the recognition properties of the oligonucleotides.

### Example 2

### Preparation of DNA-Modified ROMP Polymer Nanoparticle Assemblies

In this Example, the oligonucleotide modified ROMP polymer conjugate described in Example 1 was utilized to form nanoparticle assemblies. When a PBS buffer solution of Hybrid-I (12 μL of 8.3 μM in DNA) is mixed with a PBS buffer solution of 13 nm gold nanoparticles (260 μL of 9.7 niM in particle) modified with complementary DNA strands,² three-dimentional particle aggregates is signaled by the diagnostic shift in the surface plasmon resonance of the particles (from 520 nm to 570 nm, FIG. 3C) and a corresponding change in color (from red to purple).^{2,3} Transmission electron microscopy studies reveal a high networked aggregate (FIG. 3D). Control experiments in which a solution of the same nanoparticles was mixed with a buffered solution of Hybrid-II (which is non-complementary) resulted in no aggregate formation under nearly identical conditions.

A. Preparation Of 13 nm Gold Particles

Oligonucleotide-modified 13 nm Au particles were prepared by literature methods (˜110 oligonucleotides/particle).¹⁸⁻²⁰ Gold colloids (13 nm diameter) were prepared by reduction of HAuCl₄ with citrate as described by Frens, Nature Phys. Sci., 241, 20 (1973) and Grabar, Anal. Chem., 67, 735 (1995). Briefly, all glassware was cleaned in aqua regia (3 parts HCl, 1 part HNO₃), rinsed with nanopure H₂O, then oven dried prior to use. HAuCl₄ and sodium citrate were purchased from Aldrich Chemical Company. An aqueous solution of HAuCl₄ (1 mM, 500 mL) was brought to a reflux while stirring, and then aqueous trisodium citrate (50 mL of a 38.8 mM solution) was added quickly, which resulted in a change in solution color from pale yellow to deep red. After the color change, the solution was refluxed for an additional fifteen minutes, allowed to cool to room temperature, and subsequently filtered through a 0.45 micron nylon filter (Micron Separations Inc.). The resulting Au colloids solution were characterized by UV-Vis spectroscopy using a Hewlett Packard 8452A diode-array spectrophotometer and by Transmission Electron Microscopy (TEM) using a Hitachi 8100 transmission electron microscope. A typical solution of 13-nm diameter gold particles exhibited a characteristic surface plasmon band centered at 518-520 nm. Gold particles with diameters of 13 nm will produce a visible color change when aggregated with target and probe oligonucleotide sequences in the 10-72 nucleotide base pairs range.

B. Synthesis Of Oligonucleotides

Oligonucleotides were synthesized on a 1-micromole scale using a Milligene Expedite DNA synthesizer in single column mode and phosphoramidite chemistry. Eckstein, F. (ed.) Oligonucleotides and Analogues: A Practical Approach (IRL Press, Oxford, 1991). All solutions were purchased from Milligene (DNA synthesis grade). Average coupling efficiency varied from 98 to 99.8%, and the final dimethoxytrityl (DMT) protecting group was not cleaved from the oligonucleotides to aid in purification.

Preparation of 3′-thiol oligonucleotides. For 3′-thiol oligonucleotides, Thiol-Modifier C3 S-S CPG support was purchased from Glen Research and used in the automated synthesizer. The final dimethoxytrityl (DMT) protecting group was not removed to aid in purification. After synthesis, the supported oligonucleotide was placed in concentrated ammonium hydroxide (1 mL) for 16 hours at 55° C. to cleave the oligonucleotide from the solid support and remove the protecting groups from the bases.

After evaporation of the ammonia, the oligonucleotides were purified by preparative reverse-phase HPLC using an HP ODS Hypersil column (5 μm, 250×4 mm) with 0.03 M triethyl ammonium acetate (TEAA) eluent (pH 7) and a 1%/minute gradient of [95% CH₃CN/5% 0.03 M TEAA] at a flow rate of 1 mL/minute, while monitoring the UV signal of DNA at 254 nm. The retention time of the DMT-protected modified 12-base oligomer averages <b/c it varies with the sequences> at about 30 minutes. The DMT was subsequently cleaved by soaking the purified oligonucleotide in an 80% acetic acid solution for 30 minutes followed by evaporation. The resulting oligonucleotide was redispersed in water (500 μL), and the solution was extracted with ethyl acetate (3×300 μL). After evaporation of the solvent, the oligonucleotide (10 OD's) was redispersed in 100 μL of a [0.04 M DTT, 0.17 M phosphate] buffer solution (pH 8) and kept overnight at 50° C. to cleave the 3′ disulfide linkage. Aliquots of this solution (<10 OD's) were purified through a desalting NAP-5 column. The amount of oligonucleotide was determined by absorbance at 260 nm. Purity was assessed by ion-exchange HPLC using a Dionex Nucleopac PA-100 column (250×4 mm) with 10 mM NaOH (pH 12) eluent and a 2%/minute gradient of [10 mM NaOH, 1 M NaCl] at a flow rate of 1 mL/minute while monitoring the UV signal of DNA at 254 nm.

Preparation of 5′-alkylthiol modified oligonucleotides. 5′-alkylthiol modified oligonucleotides were prepared using the following syringe method protocol: 1) a CPG-bound, detritylated oligonucleotide was synthesized on an automated DNA synthesizer (Expedite) using standard procedures; 2) the CPG-cartridge was removed and disposable syringes were attached to the ends; 3) 200 μL of a solution containing 20 μmole of 5-Thiol-Modifier C6-phosphoramidite (Glen Research) in dry acetonitrile was mixed with 200 μL of standard “tetrazole activator solution” and, via one of the syringes, introduced into the cartridge containing the oligonucleotide-CPG; 4) the solution was slowly pumped back and forth through the cartridge for 10 minutes and then ejected followed by washing with dry acetonitrile (2×1 mL); 5) the intermediate phosphite was oxidized with 700 μL of a 0.02 M solution of iodine in THF/pyridine/water (30 seconds) followed by washing with acetonitrile/pyridine (1:1; 2×1 mL) and dry acetonitirile. The tritylated oligonucleotide derivative was then isolated and purified as described above for the 3′-alkylthiol oligonucleotides. The trityl protecting group was then cleaved by adding 15 μL (for 10 OD's) of a 50 mM AgNO₃ solution to the dry oligonucleotide sample for 20 minutes, which resulted in a milky white suspension. The excess silver nitrate was removed by adding 20 uL of a 10 mg/mL solution of DTT which immediately formed a yellow precipitate (within five minutes of reaction time) that was removed by centrifugation. Aliquots of the oligonucleotide solution (<10 OD's) were then transferred onto a desalting NAP-5 column for purification. The final amount and the purity of the resulting 5′ alkylthiol oligonucleotides were assessed using the techniques described above for 3′ alkylthiol oligonucleotides. Two major peaks were observed by ion-exchange HPLC with retention times of 19.8 minutes (thiol peak, 16% by area) and 23.5 minutes (disulfide peak, 82% by area).

C. Attachment of Oligonucleotides to Gold Particles

A 1-mL aliquot of the gold colloids solution (17 nM) in water was mixed with excess (3.68 M) thiol-oligonucleotide (22 bases in length) in water, and the mixture was allowed to stand for 12-24 hours at room temperature. Then, the solution was brought into a [0.1 M NaCl, 10 mM phosphate] buffer solution (pH 7) and allowed to stand for 40 hours. This “aging” step was designed to increase the surface coverage by the thiol-oligonucleotides and to displace oligonucleotide bases from the gold surface. The solution was next centrifuged at 14,000 rpm in an Eppendorf Centrifuge 5414 for about 25 minutes to give a very pale pink supernatant containing most of the oligonucleotide (as indicated by the absorbance at 260 nm) along with 7-10% of the colloidal gold (as indicated by the absorbance at 520 nm), and a compact, dark, gelatinous residue at the bottom of the tube. The supernatant was removed, and the residue was resuspended in about 200 μL of buffer [10 mM phosphate, 0.1 M NaCl] and recentrifuged. After removal of the supernatant solution, the residue was taken up in 1.0 mL of another buffer [10 mM phosphate, 0.3 M NaCl, 0.01% NaN₃]. The resulting red master solution was stable (i.e., remained red and did not aggregate) on standing for months at room temperature, on spotting on silica thin-layer chromatography (TLC) plates (see Example 4), and on addition to [1 M NaCl, 10 mM MgCl₂] solution, or solutions containing high concentrations of salmon sperm DNA.

### Example 3

### Synthesis Of DNA-Modified ROMP Block Co-Polymer

This Example describes the preparation of a ROMP block co-polymer (poly2-block-poly4) modified with oligonucleotides. See FIG. 2 for the chemical structures of the monomers and intermediates.

Synthesis of 4. Compound 4 (940 mg, 2.79 mmol, 75%) was synthesized according to the procedure outlined for the synthesis of B. Reaction scale: exo-5-norbornene-2-ol (500 mg, 4.54 mmol), Na metal (150 mg, 6.52 mmol), and 3-(bromopropyl)-ferrocene (1.15 g, 3.74 mmol). ¹H NMR (CDCl₃): δ 1.32-1.75 (m, 4H, 3- and 7-norbornenyl-H), 1.80 (m, 2H,CH₂—CH₂-Cp), 2.40 (t, 2H, CH₂-Cp), 2.81 (b, 1H, 1-norbornenyl-H), 2.89 (m, 1H, 4-norbornenyl-H), 3.40-3.50 (m, 3H, 2-norbornenyl-H and CH₂), 4.00-4.10 (m, 4H, Cp-H), 4.11 (s, 5H, Cp-H), 5.92 (m, 1H, 6-norbornenyl-H), 6.19 (m, 1H, 5-norbornenyl-H). ¹³C NMR (CDCl₃): δ 26.35, 31.45, 34.67, 40.57, 46.18, 46.59, 67.29, 68.27, 68.71, 68.97, 80.50, 133.47, 140.82. HREIMS: Calcd for C₂₀H₂₄FeO: 336.1177. Found: 336.1179.

Synthesis of poly2-block-poly4. In an inert atmosphere glovebox, 2 (95 mg, 0.29 mmol) was weighed into a 25-mL round bottom flask equipped with a magnetic stirring bar. Dry THF (4 mL) was added, followed by the injection of a solution of catalyst 1 (12.5 mg, 0.015 mmol) in dry THF (0.5 mL). The mixture was stirred for 60 min, after which time a solution of 4 (51 mg, 0.15 mmol) in dry THF (1 mL) was injected. After a further 60 min, the solution was removed from the dry box and the polymerization was terminated with ethyl vinyl ether (1 mL). The polymer (poly2-block-poly4, 140 mg, 96%) was isolated by pouring the mixture into pentane, filtering, and repeatedly washing with fresh pentane (4×20 mL). GPC (THF): Mₙ=12,500, PDI=1.6.

Modification of poly2-block-poly4 with 3. Poly2 (100 mg) was dissolved in dry THF (5 mL). N,N-Diisopropylethylamine (100 μL) and 3 (70 mg, 0.29 mmol) were added and the mixture was stirred at room temperature for 2 h. The modified block copolymer was isolated by pouring the mixture into pentane, filtering, and repeatedly washing with fresh pentane (4×20 mL). ³¹P NMR (CDCl₃): δ 149.2.

Attachment of DNA to Modified poly2-block-poly4. The attachment of DNA to modified poly2-block-poly4 was accomplished on CPG supports using conventional phosphoramidite chemistry⁴ and an automated DNA synthesizer (Expedite), except CHCl₃ was used as a solvent during the coupling step instead of acetonitrile, and purification was accomplished using ultrafiltration using a Centricon-50 instead of HPLC.

Conclusion: Given the exceptional functional group tolerance of catalyst 1 in ROMP, it has been hypothesized that this chemistry could be extended to block copolymers of 2 with a number of norbornene monomers, thereby imparting tailorable functionality to branched DNA structures. The strategy for the synthesis of block copolymer branched DNA is outlined in FIG. 4. In these experiments, monomer 2 is mixed with a catalytic amount of 1 in dry THF. After 1 h, the polymerization of the first block was determined to be complete by ¹H NMR spectroscopy. Subsequent addition and polymerization of a second norbornene monomer, followed by the injection of ethyl vinyl ether to terminate the reaction, yielded block copolymers with the desired structure. When the norbornenyl-modified ferrocene 4 was used as a second block, post-polymerization modification with 3 and solid phase DNA synthesis led to the isolation of Hybrid III and Hybrid IV. Electrochemical measurements confirm the presence of the ferrocenes (Fc) in these hybrids (E½=33 mV vs Fc/Fc⁺, FIG. 5A). When PBS-buffered solutions of these hybrids were mixed, thermally reversible aggregate formation was again observed. Also, a sharp melting transition was observed, consistent with our proposed structure (FIG. 5B).

The data in this example illustrates that the post-polymerization modification of ROMP polymers and block copolymers with DNA can lead to DNA/polymer hybrid materials with a number of interesting properties associated with the hybrid structure. The initial experiments described herein reveal that the recognition properties of the DNA strands are not adversely affected by attachment to the polymer. These new structures can be prepared with properties and function that depend upon the choice of ROMP monomer and DNA branch sites. Since the synthesis of block copolymers of 2 with other norbornenyl-modified compounds is a facile process, the isolation of other novel and potentially useful macromolecular hybrid materials should be readily accomplished by utilizing variations of the strategy presented herein.

### Example 4

### Preparation of Redox Active DNA-Modified ROMP Block Copolymer

The above Examples demonstrated the chemical attachment of several DNA strands to a well defined organic polymers and block copolymers derived from ring-opening metathesis polymerization (ROMP).²² In this approach, the commercially available metathesis catalyst Cl₂(PCy3)₂Ru═CPh (1) was used to polymerize a novel norbornenyl-modified alcohol with a diphenylacetylene spacer (2). Post-polymerization modification with 2-cyanoethyl diisopropylchlorophosphoramidite (3) led to the isolation of a polymer ready for coupling to DNA using standard solid phase techniques. Significantly, polymers modified with complementary strands hybridized to form reversible aggregates with very sharp melting characteristics.

Oligonucleotides with electrochemically active molecules have been exploited for DNA detection.²³⁻²⁵ Electrochemical detection of single-base mismatches has recently been reported using two types of ferrocene-containing oligonucleotides as dual-signaling probes.²⁵ In this Example, both the generality and the utility of a block copolymer strategy for electrochemical DNA detection is demonstrated where sensitivity can be significantly amplified with cooperative binding and multiple-layer assembly. Block copolymers with highly tailorable redox-activity can be readily coupled to DNA strnads using a post-polymerization modification approach. Specifically, both diblock and triblock copolymers can be employed to increase the number of indicators for unique DNA strands, effectively “tagging” each strand with a unique electrochemical “barcode”. Finally, DNA detection utilizing the inventive hybrid structures is demonstrated.

Syntheses of Block Copolymers. Blockcopolymers (Hybrid I-Hybrid IV) of DNA and redox active molecules (4 or 5) were synthesized from ROMP (FIG. 6). DNA sequences are given in Table 1.

TABLE 1
DNA sequences of Hybrid Polymers.

| | Polymer Pre- | |
|---|---|---|
| Hybrid | cursor (Ratio) | Sequence |
| I | poly2-poly4 | 3′TTA TAA CTA TTC CTA T₃5′ |
| | (17:9) | |
| II | poly2-poly4 | 3′TAG GAA TAG TTA TAA T₃5′ |
| | (17:9) | |
| III | poly2-poly5 | 3′TAG GAC TTA CGC TAT T₃5′ |
| | (17:9) | |
| IV | poly2-poly5 | 3′ATA GCG TAA GTC CTA T₃5′ |
| | (17:9) | |
| V | poly2-poly4- | 3′TAG GAC TTA CGC TAT T₁₀5′ |
| | poly6 (17:5:10) | |
| XI | poly2-5-7 | 3′TTA TAA CTA TTC CTA T₁₀5′ |
| | (17:10:5) | |

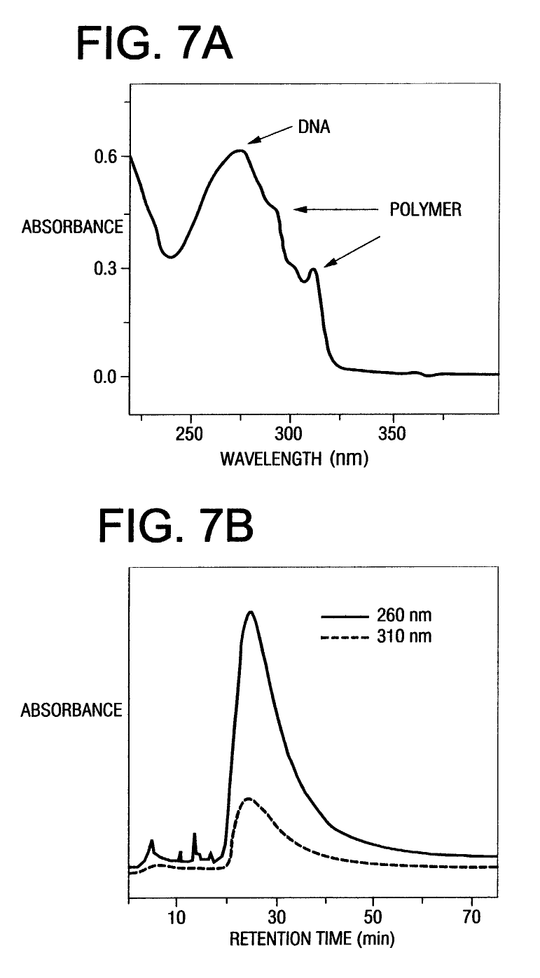

The presence of the oligonucleotides side chains help to solubilize the resulting DNA/polymer hybrids in aqueous solution. The number of DNA strands attached to each hybrid was estimated from the UV-Vis absorption spectra of the polymers in water (FIG. 7A). Based on the extinction coefficients of DNA (λₘₐₓ=260 nm) and diphenylacetylene (λₘₐₓ=310 nm), we determined that there are on average five DNA strands per a single polymer chain, which translates to ˜30% occupation of the total DNA coupling sites (17). Ion exchange HPLC was performed on purified Hybrid I (FIG. 7B). One major peak at 25 min was observed at both λₘₐₓ=260 nm and 310 nm which further demonstrates that DNA is indeed coupled to polymer backbone.

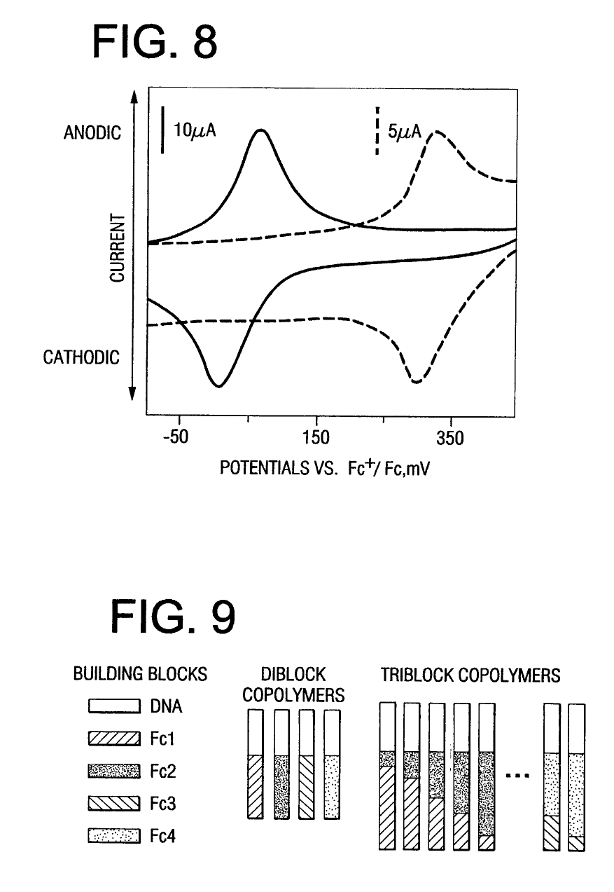

The redox potential of the DNA/polymer hybrids can be tailored by using ferrocene derivatives with electron donating or withdrawing substituent. Two different norbornenyl-modified ferrocene derivatives, 4 and 5, were chosen because of their redox potential difference. Since the carbonyl group attached to the ferrocene ring of 5, it oxidizes at higher potential than 4 (ΔE½˜300 mV). Both monomers were polymerizable using 1 and incorporated into the blockcopolymer structure to yield poly2-poly4 and poly2-poly5. Those polymer precursors were readily coupled to 5′ end of oligonucleotides to yield Hybrid I-Hybrid IV as described in FIG. 6. Electrochemical measurements were carried out on thin films of these hybrids by casting them on Au electrodes. Cyclic voltammetry (CV) revealed stable and reversible waves associated with oxidation and reduction of the ferrocene blocks of the hybrid materials. As shown in FIG. 8, the E_{1/2} value for Hybrid I was found to be 30 mV (versus Fc/Fc⁺), while an E_{1/2} of Hybrid III was 330 mV (versus Fc/Fc⁺). These values are consistent with redox potentials expected from the monomers, 4 and 5.

Synthesis of 11-(bicyclo[2.2.1]hept-5-en-2-exo-yloxy)-1-ferrocenyl undecan-1-one (5). In an inert atmosphere glovebox, exo-5-norbornene-2-ol (500 mg, 4.54 mmol) was weighed into a 50-mL Schlenk flask. THF (15 mL) was added, and the solution was stirred while oil-free sodium metal (150 mg, 6.52 mmol) was added. The reaction flask was then taken out of the glovebox, attached to a water-cooled condenser, refluxed for 12 h under a nitrogen bubbler, and allowed to cool to room temperature. In a separate 100-mL Schlenk flask, 11-bromoundecanoyl-ferrocene (2.00 g, 4.62 mmol) was dissolved in dry THF (15 mL) under nitrogen. The cooled solution of deprotonated exo-5-norbornen-2-ol was then transferred via cannula to the 11-bromoundecanoyl-ferrocene solution with vigorous stirring. The flask was capped with a water-cooled condenser and the resulting mixture was refluxed for an additional 12 h under a nitrogen bubbler. Upon cooling to room temperature, the reaction mixture was poured into ether (50 mL) and washed successively with water (50 mL), 0.1 M NaOH (50 mL), 1.0 M HCl (50 mL), and brine (50 mL). The organic layer was collected, dried over sodium sulfate, and filtered into a 500-mL round bottom flask. The solvent from the filtrate was removed on a rotary evaporator. Column chromatography of the residue on silica gel with 10% ethyl acetate in hexanes as the eluent gave the desired product 5 as a dark red oil. ¹H NMR (CDCl₃): δ 1.24-1.73 (m, 20H, 3- and 7-norbornenyl-H and 8×CH₂), 2.70 (t, 2H, CH₂—C═O), 2.79 (b, 1H, 1-norbornenyl-H), 2.87 (b, 1H, 4-norbornenyl-H), 3.36-3.47 (m, 3H, 2-norbornenyl-H and CH₂—O), 4.20 (s, 5H, Cp-H), 4.49 (m, 2H, Cp-H), 4.78 (m, 2H, Cp-B), 5.91 (m, 1H, 6-norbornenyl-H), 6.18 (m, 1H, 5-norbornenyl-H). ¹³C NMR (CDCl₃): δ 24.83, 26.49, 29.6-29.8 (b, 6 Cs), 30.27, 34.62, 39.96, 40.54, 46.14, 46.58, 69.51, 69.91, 72.28, 79.37, 80.35, 133.48, 140.75, 204.92. HREIMS: Calcd for C₂₈H₃₈FeO₂: 462.222. Found: 462.224.

Synthesis of (3-bromopropanoyl)dibromoferrocene. Under ambient conditions, AICl₃ (767 mg, 5.75 mmol) was quickly weighed into a 50-mL Schlenk flask containing a magnetic stirbar. The flask was then evacuated and placed under nitrogen. Next, methylene chloride (25 mL) was cannula-transferred into the flask and the reaction mixture was cooled to 0° C. while stirring. After 10 minutes, 3-bromo-propionyl chloride (1.025 g, 5.98 mmol) was added to the reaction mixture via syringe. The resulting mixture was stirred for an additional 20 minutes. In another 50-mL Schlenk flask 1,1′-dibromoferrocene[ref] (1.875 g, 5.45 mmol) was added, and the flask was evacuated and placed under N₂. Methylene chloride (10 mL) was added and the resulting solution was cannula transferred into the stirring AlCl₃ mixture, at which point the color of the reaction turned dark purple. The reaction mixture was removed from the ice bath and allowed to stir for 12 h under nitrogen. Next, NaHCO₃ (30 mL of a saturated aqueous solution) was slowly added to the reaction via syringe and stirred for an additional 15 minutes. The reaction mixture was then poured into water and extracted with methylene chloride (2×50 mL). The organic portions were combined and washed with water (in 50 mL portions) until the aqueous washes become pH-neutral, dried over sodium sulfate, and filtered into a 500-mL round bottom flask. The solvent was removed from the filtrate and evaporated from the crude residue on a rotary evaporator. Column chromatography of the reaction residue on silica gel with 20% ethyl acetate in hexanes as the eluent gave the desired product as mixture of two isomers (1.502 g, 3.14 mmol, 57.6%) as a red oil. ¹H NMR (CDCl₃): δ 3.16-3.59 (m, 2H, CH₂—C═O), 3.71-3.96 (m, 2H, CH₂—Br), 4.21-4.33 (m, 2H, Cp-H), 4.48-4.58 (m, 2H, Cp-H), 4.77-4.82 (m, 2H, Cp-H), 5.00-5.03 (b, 1H, Cp-H). ¹³C NMR (CDCl₃): 25.87, 25.98, 38.77, 42.52, 42.72, 44.02, 44.21, 69.37, 71.04, 71.59, 71.81, 71.88, 72.14, 72.21, 73.33, 73.56, 74.04, 74.55, 74.64, 75.39, 77.17, 77.33, 77.42, 77.68, 78.37, 78.80, 79.07, 79.15, 199.14, 199.35.

Synthesis of (3-bromopropyl)dibromoferrocene. For the sake of convenience the reaction was done open to air using excess LAH and AlCl₃. LAH (395 mg, 10.4 mmol) and AlCl₃ (712 mg, 5.34 mmol) were quickly weighed into a 50-mL Schlenk flask which was then equipped with a magnetic stirbar and an addition funnel. Dry ether (15 mL) was cannula transferred into the addition funnel and slowly added to the stirring mixture. (3-bromopropanoyl)dibromoferrocene (1.245 g, 2.6 mmol) was weighed into a 50-ml Schlenk flask, dissolved in dry ether (15 mL). This solution was then pipetted into the addition funnel and added to the stirring LAH and AlCl₃ reaction mixture over a period of 30 minutes. Next, the solution was stirred for 20 minutes, neutralized with water (20 mL) slowly added over 10 minutes, and stirred for an additional 10 minutes. The contents of the Schlenk flask were then poured over water and washed with ether (2×50 mL). The organic layers were combined and washed twice more with 50 mL portions of water and dried over Na₂SO₄. The solution was filtered, and the solvent was removed with a rotary evaporator. The crude product was chromatographed on a silica gel column with 5% methylene chloride in hexanes as eluent to give the desired mixture of two isomers (935 mg, 2.01 mmol, 77.3%) as an orange oil. ¹H NMR (CDCl₃): δ 2.05 (b, 2H, CH₂—CH₂—Br), 2.49-2.62 (2s, 2H, CH₂-Cp), 3.42-3.55 (2s, 2H, CH₂—Br), 4.06-4.13 (m, 4H, Cp-H), 4.36-4.42 (m, 3H, Cp-H). ¹³C NMR (CDCl₃): 25.73, 26.26, 26.96, 33.28, 33.50, 33.64, 33.76, 44362, 68.71, 69.74, 69.97, 70.53, 70.77, 70.99, 71.21, 72.18, 72.45, 72.89, 73.28, 73.68, 73.77, 78.19, 79.05, 88.34.

Synthesis of 5-exo-[(3-dibromoferrocenylpropyl)oxy]bicyclo[2.2.1]hept-2-ene (6). In an inert atmosphere glovebox, exo-5-norbornene-2-ol (257 mg, 2.33 mmol) was weighed into a 50-mL Schlenk flask. THF (15 mL) was added, and the solution was stirred while oil-free sodium metal (108 mg, 4.70 mmol) was added. The reaction flask was then taken out of the glovebox, attached to a water-cooled condenser, refluxed for 12 h under a nitrogen bubbler, and allowed to cool to room temperature. In a separate 100-mL Schlenk flask, (3-bromopropyl)dibromoferrocene (876 mg, 1.88 mmol) was dissolved in dry THF (15 mL) under nitrogen. The cooled solution of deprotonated exo-5-norboren-2-ol was then transferred cannula to the (3-bromopropyl)dibromoferrocene solution with vigorous stirring. The flask was capped with a water-cooled condenser and the resulting mixture was refluxed for an additional 12 h under a nitrogen bubbler. Upon cooling to room temperature, the reaction mixture was poured into ethyl acetate (50 mL) and washed successively with water (2×50 mL). The organic layer was collected, dried over sodium sulfate, and filtered into a 500-mL round bottom flask. The solvent from the filtrate was removed on a rotary evaporator. Column chromatography of the residue on silica gel with 45% methylene chloride in hexanes as the eluent gave the desired mixture of two isomers as an orange oil. ¹H NMR (CDCl₃): δ 1.26-1.70 (m, 4H, 3- and 7-norbornenyl-H), 1.77-1.78 (m, 2H, CH₂—CH-Cp), 2.38-2.51 (2t, 2H, CH₂-Cp), 2.81 (b, 1H, 1-norbornenyl-H), 2.88 (b, 1H, 4-norbornenyl-H), 3.47-3.48 (m, 3H, 2-norbornenyl-H and CH₂O), 4.03-4.13 (m, Cp-H), 4.30-4.40 (m, Cp-H), 5.93 (m, 1H, 6-norbornenyl-H), 6.19 (m, 1H, 5-norbornenyl-H). ¹³C NMR (CDCl₃): 24.29, 24.31, 25.20, 30.57, 31.10, 34.60, 34.63, 34.66, 40.51, 46.14, 46.50, 46.52, 46.59, 68.36, 68.49, 68.53, 68.62, 69.43, 69.47, 69.73, 69.76, 70.41, 70.57, 70.80, 71.83, 72.15, 72.71, 72.76, 72.78, 73.16, 73.36, 73.58, 78.08, 78.97, 78.98, 80.47, 80.53, 88.83, 88.86, 89.85, 133.39, 133.42, 140.79.

Synthesis of toluene-4-sulfonic acid 2-{2-[2-(bicyclo[2.2.1]hept-5-en-2-yloxy)-ethoxyl]-ethoxy}-ethyl ester. In an inert atmosphere glovebox, exo-5-norbornene-2-ol (500 mg, 4.54 mmol) was weighed into a 50-mL Schlenk flask. Dry dioxane (15 mL) was added, and the solution was stirred while oil-free sodium metal (150 mg, 6.52 mmol) was added. The reaction flask was then taken out of the glovebox, attached to a water-cooled condenser, refluxed for 12 h under a nitrogen bubbler, and allowed to cool to room temperature. In a separate 50-mL Schlenk flask, tris(ethylene glycol)-αωbis(p-tosylate) (2.10 g, 4.58 mmol) was dissolved in dry dioxane (15 mL) under nitrogen. This solution was then stirred vigorously while the cooled solution of deprotonated exo-5-norbornen-2-ol was added via cannula. The flask was capped with a water-cooled condenser and refluxed for an additional 48 h under a nitrogen bubbler. Upon cooling to room temperature, the reaction mixture was poured into ethyl acetate (50 mL) and washed successively with water (3×50 mL) and brine (50 mL). The organic layer was collected, dried over sodium sulfate, and filtered into a 500-mL round bottom flask. The solvent from the filtrate was removed on a rotary evaporator. Column chromatography of the residue on silica gel with 40% ethyl acetate in hexanes as the eluent gave the desired product 7 (335 mg, 0.84 mmol, 19%) as a clear oil. ¹H NMR (CDCl₃): δ 1.26-1.67 (m, 4H, 3- and 7-norbornenyl-H), 2.45 (s, 3H, Ph-CH₃), 2.78 (b, 1H, 1-norbornenyl-H), 2.88 (b, 1H, 4-norbornenyl-H), 3.49-3.65 (m, 9H, 2-norbornenyl-H and CH₂CH₂—O), 3.70 (m, 2H, CH₂—CH₂—OSO₂), 4.17 (m, 2H, CH₂—OSO₂), 5.91 (m, 1H, 6-norbornenyl-H), 6.18 (m, 1H, 5-norbornenyl-H), 7.35 (d, 2H, Ph-H), 7.81 (d, 2H, Ph-H). ¹³C NMR (CDCl₃): δ 21.99, 34.73, 40.67, 46.20, 46.64, 68.77, 68.93, 69.48, 70.82, 71.00, 71.01, 80.96, 128.08, 129.89, 133.12, 133.22, 140.75, 144.81. Anal.: Calcd for C₂₀H₂₈O₆S: C, 60.58; H, 7.12. Found: C, 60.75; H, 6.99.

Synthesis of 5-{2-[2-(2-ferrocenyl-oxy-ethoxy)-ethoxy]-ethoxy}-bicyclo[2.2.1hept-2-ene (7). In an inert atmosphere glovebox, ferrocene acetate (300 mg, 1.23 mmol) was weighed into a 50-mL Schlenk flask. Degassed absolute ethanol (15 mL) was added, and the solution was stirred while oil-free KH (95 mg, 2.34 mmol) was added. The reaction flask was then taken out of the glovebox, attached to a water-cooled condenser, refluxed for 45 min under a nitrogen bubbler, and allowed to cool to room temperature. In a separate 50-mL Schlenk flask, toluene-4-sulfonic acid 2-{2-12-(bicyclo[2.2.1]hept-5-en-2-yloxy)-ethoxy]-ethoxy}-ethyl ester (340 mg, 0.86 mmol) was dissolved in degassed ethanol (100%, 15 mL) under nitrogen and then transferred via cannula to the stirred solution of ferrocene acetate and KH. The flask was capped with a water-cooled condenser and refluxed for an additional 12 h under a nitrogen bubbler. Upon cooling to room temperature, the reaction mixture was poured into ethyl acetate (50 mL) and washed successively with water (3×50 mL) and brine (50 mL). The organic layer was collected, dried over sodium sulfate, and filtered into a 500-mL round bottom flask. The solvent from the filtrate was removed on a rotary evaporator. Column chromatography of the residue on silica gel with 20% ethyl acetate in hexanes as the eluent gave the desired product 7 (230 mg, 0.54 mmol, 63%) as a dark red oil. ¹H NMR (CDCl₃): δ 1.26-1.69 (m, 4H, 3- and 7-norbornenyl-H), 2.79 (b, 1H, 1-norbornenyl-1), 2.90 (b, 1H, 4-norbornenyl-H), 3.50-3.82 (m, 13H, 2-norbornenyl-H and, CH₂—CH₂—O), 3.98 (m, 2H, Cp-H), 4.13 (m, 2H, Cp-H), 4.21 (s, 5H, Cp-H), 5.92 (m, 1H, 6-norbornenyl-H), 6.18 (m, 1H, 5-norbornenyl-H). ¹³C NMR (CDCl₃): 34.74, 40.69, 46.23, 46.66, 55.77, 62.13, 68.70, 68.81, 70.05, 70.10, 71.05, 71.10, 80.97, 126.37, 133.26, 140.75. Anal.: Calcd for C₂₃H₃₀FeO₄: C, 64.80; H, 7.09. Found: C, 64.77; H, 7.27.

Syntheses of poly2-poly5. The notation “poly2-poly5” and “poly2-block-poly5” are used interchangeably and refer to the same polymer. In an inert atmosphere glovebox, 2 (100 mg, 0.30 mmol) was weighed into a 25-mL round bottom flask equipped with a magnetic stirring bar. Dry THF (4 mL) was added, followed by a solution of catalyst 1 (15.0 mg, 0.018 mmol) in dry THF (0.5 mL). The mixture was stirred for 60 min, after which time a solution of 5 (60 mg, 0.13 mmol) in dry THF (0.5 mL) was injected into the mixture. After a further 45 min, the reaction was removed from the dry box and the polymerization was terminated with ethyl vinyl ether (1 mL). The polymer (poly2-poly5, 148 mg, 93%) was isolated by pouring the mixture into pentane, filtering, and repeatedly washing with fresh pentane (4×20 mL). GPC (THF): Mₙ=9,500; PDI=1.2.

Modification of poly2-poly5 with 3. Poly2-poly5 (100 mg) was dissolved in dry THF (5 mL). Diisopropylethylamine (200 μL) and 3 (100 mg, 0.41 mmol) were added and the mixture was stirred at room temperature for 2 h. The mixture was poured into pentane (100 mL), filtered, washed with fresh pentane (4×20 mL), and concentrated to dryness to yield the desired product which was dissolved in CDCl₃ for ³¹P NMR analysis and then used directly in the next step. ³¹P NMR (CDCl₃): δ 149.2.

Attachment of DNA to Modified poly2-poly5. The attachment of DNA to modified poly2-poly5 was accomplished using the syringe synthesis technique. CDCl₃ was used as a solvent during the coupling of modified poly2-poly5 to the oligonucleotides on CPG support instead of acetonitrile. After synthesis, the supported polymer was placed in 1 mL of ammonium hydroxide at 60° C. for 16 h to remove the protecting groups from bases and cleave the polymers and failure DNA stands from the support. Purification was accomplished using ultrafiltration with a Centricon-100 instrument. High-performance liquid chromatography (HPLC) was performed using a HP series 1100 HPLC equipped with an ion-exchange column with 10 mM NaOH eluent and a 2%/min gradient of [10 mM NaOH, 2 M NaCl] at a flow rate of 1 mL/min, while monitoring the UV absorbance at 260 nm and 310 nm.

### Example 5

### Synthesis of DNA-Modified ROMP Triblock Co-Polymers

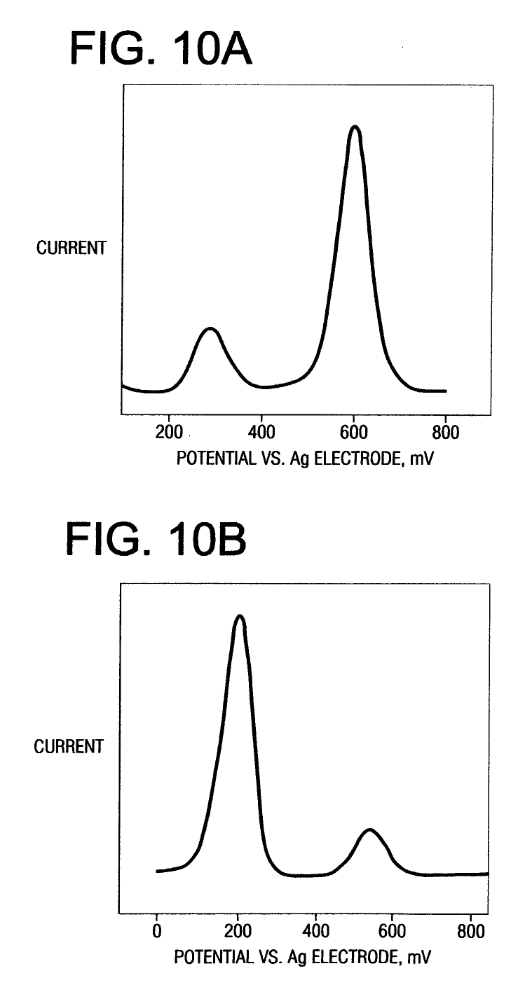

Based on the blockcopolymer strategy discussed above, this Example illustrates the preparation of DNA-modified ROMP triblock co-polymer one can incorporate about four different indicators. To increase the number of indicators thus to use their redox potentials as a type of barcode, triblock copolymers were synthesized to contain two different ferrocenyl derivatives, 4 and 6. By adjusting the ratio between any two redox active blocks, one can generate many different indicators rather than the maximum two in the case of diblock copolymers where each redox-active monomer can only be used once (Scheme 2). Triblock copolymer precursors, poly2-poly4-poly6 were synthesized by successively growing 2, 4 and 6 onto a propagating ROMP chain. For proof-of-concept experiments, triblock copolymers with approximately 1:2 and 2:1 ratios of 4 and 6 were synthesized. Gel permeation chromatography (GPC) data of these polymers showed a single peaks, indicating that the three components are in one entity. These precursors were coupled to DNA to generate DNA triblockcopolymers. Cyclic voltamograms of the polymers exhibit two peaks at 30 mV and 330 mV (versus Fc/Fc⁺) (FIG. 10A). Ratios of peak areas are 1:4.2 and X/X, respectively.

Syntheses of triblock copolymers, poly2-poly4-poly6. In an inert atmosphere glovebox, 2 (45.4 mg, 0.14 mmol) was weighed into a 25-mL round bottom flask equipped with a magnetic stirring bar. Dry THF (3 mL) was added, followed by a solution of catalyst 1 (6.6 mg, 0.008 mmol) in dry THF (0.5 mL). The mixture was stirred for 2 h, after which time a solution of 4 (26.5 mg, 0.079 mmol) in dry THF (0.5 mL) was injected into the mixture. The mixture was stirred for 12 h, then a solution of 6 (20 mg, 0.040 mmol) in dry THF (0.5 mL) was injected into the mixture. The reaction was stirred for 2 h, removed from the dry box, and the polymerization was terminated with ethyl vinyl ether (1 mL). The polymer (poly2-poly4-poly6, 72 mg, 78%) was isolated by adding the mixture dropwise into a stirred solution of pentane, filtering, and repeatedly washing with fresh pentane (4×20 mL).

Modification of poly2-poly4-poly6 with 3. Poly2-poly4-poly6 (50 mg) was dissolved in dry THF (5 mL). Diisopropylethylamine (100 μL) and 3 (50 mg, 0.21 mmol) were added and the mixture was stirred at room temperature for 2 h. The mixture was poured into pentane (100 mL), filtered, washed with fresh pentane (4×20 mL), and concentrated to dryness to yield the desired product which was dissolved in CDCl₃ for ³¹P NMR analysis and then used directly in the next step. ³¹P NMR (CDCl₃): δ 149.2.

Attachment of DNA to Modified poly2-poly4-poly6. The attachment of DNA to modified poly2-poly4-poly6 was accomplished using the syringe synthesis technique. CDCl₃ was used as a solvent during the coupling of modified poly2-poly4-poly6 to the oligonucleotides on CPG support instead of acetonitrile. After synthesis, the supported polymer was placed in 1 mL of ammonium hydroxide at 60° C. for 16 h to remove the protecting groups from bases and cleave the polymers and failure DNA stands from the support. Purification was accomplished using ultrafiltration with a Centricon-100 instrument. High-performance liquid chromatography (HPLC) was performed using a HP series 1100 HPLC equipped with an ion-exchange column with 10 mM NaOH eluent and a 2%/min gradient of [10 mM NaOH, 2 M NaCl] at a flow rate of 1 mL/min, while monitoring the UV absorbance at 260 nm and 310 nm.

### Example 6

### Preparation of Redox Active DNA-Modified ROMP Random Co-Polymer

In this Example, ROMP co-polymers were prepared in a random fashion, which might improve the solubility of the hybrid molecules, as a hydrophobic block in a hybrid molecule gets longer. Random copolymers were synthesized from 5 and 7 by injecting 2, 5 and 7 at the same time rather than introducing them successively. See FIG. 6 for monomer structures. Random copolymers show expected cyclic voltamograms with distinct two redox peaks at 30 mV and 330 mV (versus Fc/Fc⁺) (FIG. 10B). Ratios of peak areas are 4.7:1 and X/X, respectively. These results demonstrate redox potentials and current ratio can be utilized as versatile indicators for multi channel DNA detection.

Syntheses of random copolymers, poly2-5-7.

For the (17:5:10) composition: In an inert atmosphere glovebox, 2 (70 mg, 0.22 mmol), 5 (29 mg, 0.063 mmol), and 7 (53 mg, 0.12 mmol) were weighed into a 25-mL round bottom flask equipped with a magnetic stirring bar. Dry THF (3 mL) was added, followed by a solution of catalyst 1 (10.3 mg, 0.013 mmol) in dry THF (0.5 mL). The mixture was stirred for 6 h, the reaction was removed from the dry box and the polymerization was terminated with ethyl vinyl ether (1 mL). The polymer (poly2-5-7, 125 mg, 82% minimum isolated yield) was isolated by adding the mixture dropwise into a stirred solution of pentane, filtering, and repeatedly washing with fresh pentane (4×20 mL). GPC (THF): Mₙ=20,700,; PDI=1.2.

For the (17:10:5) composition: In an inert atmosphere glovebox, 2 (70 mg, 0.22 mmol), 5 (58 mg, 0.125 mmol), and 7 (27 mg, 0.063 mmol) were weighed into a 25-mL round bottom flask equipped with a magnetic stirring bar. Dry THF (3 mL) was added, followed by a solution of catalyst 1 (10.3 mg, 0.013 mmol) in dry THF (0.5 mL). The mixture was stirred for 10 h, the reaction was removed from the dry box and the polymerization was terminated with ethyl vinyl ether (1 mL). The polymer (poly2-5-7, 100 mg, 65% minimum isolated yield) was isolated by adding the mixture dropwise into a stirred solution of pentane, filtering, and repeatedly washing with fresh pentane (4×20 mL). GPC (THF): Mₙ=21,300; PDI=1.2.

Modification of poly2-5-7 with 3. Poly2-5-7 (55 mg of the (17:10:5) composition) was dissolved in dry THF (2 mL). Diisopropylethylamine (100 μL) and 3 (70 mg, 0.296 mol) were added and the mixture was stirred at room temperature for 4 h. The mixture was poured into pentane (100 mL), filtered, washed with fresh pentane (4×20 mL), dried over sodium sulfate, and concentrated to dryness to yield the desired product which was dissolved in CDCl₃ for ³¹P NMR analysis and then used directly in the next step. ³¹P NMR (CDCl₃): δ 149.2.

Attachment of DNA to Modified poly2-5-7. The attachment of DNA to modified poly2-5-7 ((17:10:5) composition) was accomplished using the syringe synthesis technique. CDCl₃ was used as a solvent during the coupling of modified poly2-5-7 to the oligonucleotides on CPG support instead of acetonitrile. After synthesis, the supported polymer was placed in ACS ammonium hydroxide (1 mL) at 60° C. for 16 h to remove the protecting groups from bases and cleave the polymers and failure DNA stands from the support. Purification was accomplished using ultrafiltration performed on a Centricon-50 instrument instead of HPLC.

### Example 7

### Preparation of Materials from DNA-Modified ROMP Polymers

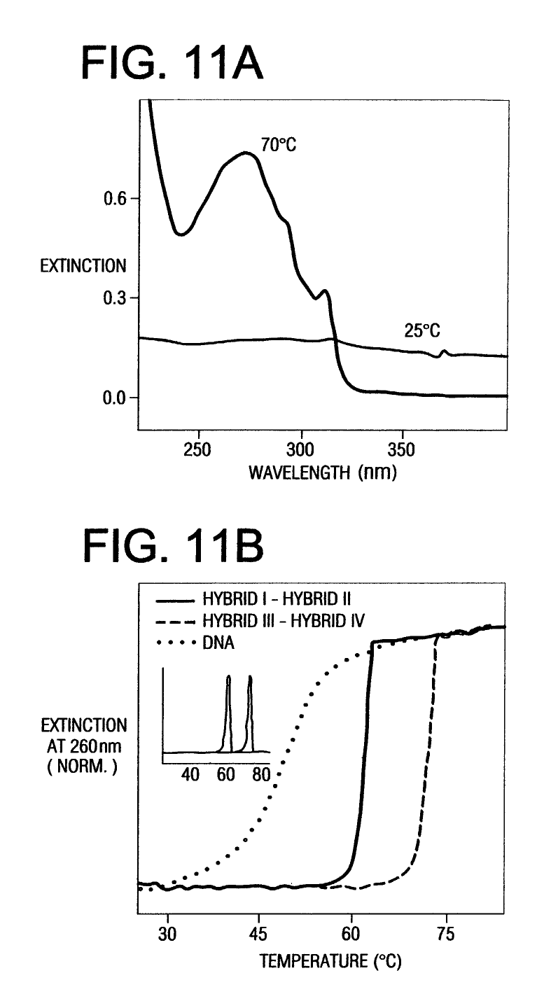

This Example illustrates that DNA recognition properties is maintained in DNA-modified ROMP block co-polymers. As outlined in Table 1 in Example 4, complementary structures Hybrid I:Hybrid III from poly2-poly4 and Hybride III:Hybrid IV from poly2-poly5) were prepared. When complementary hybrids (Hybrid I:Hybrid II, or Hybrid III:Hybrid IV) were mixed in equal amounts (0.1 mM, 20:L) in a PBS buffer (0.3 M NaCl, 10 mM phosphate, pH 7), an extended hybridization aggregate formed and precipitated from the solution in a few seconds. This demonstrates that the polymer moiety of the hybrid molecules does not hinder recognition properties of DNA. UV-Vis spectra of the aggregates showed significantly reduced DNA signal at 260 nm and increased intensity at higher wavelength due to scattering from micrometer size polymer aggregates, FIG. 11A. Upon heating the aggregate solution above DNA melting temperature, blockcopolymers were redispersed in solution evidenced by UV-Vis spectrum, FIG. 11A. The thermal denaturation curves of these aggregates were obtained by monitoring the UV-Vis spectra at 260 nm as a function of temperature, FIG. 11B. Aggregates formed form Hybrid III and IV melts at higher temperature (74° C.) than aggregates formed from Hybrid I and II (62° C.) as expected from the higher GC content of Hybrid III:IV.

Significantly, the aggregates formed from DNA blockcopolymer show higher thermal stability and extraordinarily sharper melting transition than plain duplex DNA. A melting curve of DNA duplex with same DNA sequences as Hybrid V:VI are presented in FIG. 11B for comparison. The melting temperature of aggregates formed from hybrid molecules is higher than DNA duplexes by 14° C. The higher thermal stability has been observed in DNA dendrimers,²⁶ and is consistent with multiple linkage and cooperative effect. This property is important for detecting double strand DNA.

The half maximum full widths (HMFW) of the derivatives of melting curve of hybrid molecules are 2° C. (FIG. 11B, inset). The degree of sharp melting transition has been observed in oligonucleotide-modified particles but not in DNA dendrimers. We attribute the unusual sharp melting transition of blockcopolymers to 1) high degree of cooperativity due to free directionality of DNA and 2) hydrophobic effect. The particle based detection methods also show high selectivity due to the sharp, melting characteristics.^{27,28}

### Example 8

### DNA Detection Using Hybrid Molecules

Performance of a DNA-modified ROMP blockcopolymer as DNA probes were evaluated using gold electrodes as shown in FIG. 12. In a typical experiment, thiol modified oligonucleotide a was immobilized on freshly prepared Au electrodes by applying 0.3 M PBS solution of a (1 mM) for 12 hours. Then, the electrodes were washed with water, dried with N₂, and dipped in 1 mM ethanolic solution of mercaptohexanol for 5 min. This procedure prevents nonspecific binding of hybrid molecule to gold surface. Finally, the electrodes were washed with copious amount of ethanol and water and used for DNA detection.

To evaluate the performance, synthetic target DNA a′b′ (10 nM) and a complementary DNA-polymer hybrid b (10 nM) were co-hybridized to a-modified electrodes in PBS solution for 2 hours. As a control experiment, an a-modified electrode was treated with b without target DNA in identical conditions. After washing the electrodes with PBS solution, Alternating Current (AC) voltammograms were acquired (FIG. 13). The electrode, treated with complementary target, shows desired signal while the control sample generates no detectable signal Conclusion. The above Examples demonstrate that DNA-modified ROMP blockcopolymers with various redox potentials can be readily prepared from norbornenes substituted with electrochemical tags. These polymers showed fully active DNA recognition properties and expected electrochemical properties. Importantly, DNA blockcopolymers exhibit useful properties such as sharp melting transitions and high thermal stabilities. This strategy can be extended to prepare virtually any other norbornene monomers, thereby imparting unprecedented functionality to branched DNA structures.

### REFERENCES

(1) Storhoff, J. J.; Mirkin, C. A. Chem. Rev. 1999, 99, 1849-1862.

(2) Elghanian, R.; Storhoff, J. J.; Mucic, R. C.; Letsinger, R. L.; Mirkin, C. A. Science 1997, 277, 1078-1080.

(3) Mirkin, C. A.; Letsinger, R. L.; Mucic, R. C.; Storhoff, J. J. Nature 1996, 382, 607-609.

(4) Alivisatos, A. P.; Johnson, K. P.; Peng, X.; Wilson, T. E.; Loweth, C. J.; Bruchez, M. P., Jr.; Schultz, P. G. Nature 1996, 382, 609-611.

(5) Cassell, A. M.; Scrivens, W. A.; Tour, J. M. Angew. Chem., Int. Ed. Engl. 1998, 37, 1528-1531.

(6) Niemeyer, C. M.; Burger, W.; Peplies, J. Angew. Chem., Int. Ed. Engl. 1998, 37, 2265-2268.

(7) Niemeyer, C. M. Curr. Opin. Chem. Biol. 2000, 4, 609-618.

(8) Niemeyer, C. M.; Adler, M.; Gao, S.; Chi, L. Angew. Chem., Int. Ed. Engl. 2000, 39, 3055-3059.

(9) Braun, E.; Eichen, Y.; Sivan, U.; Ben-Yoseph, G. Nature 1998, 391, 775-778.

(10) Bazin, H.; Livache, T. Nucleosides Nucleotides 1999, 18, 1309-1310.

(11) Bidan, G.; Billon, M.; Galasso, K.; Livache, T.; Mathis, G.; Roget, A.; Torres-Rodriguez, L. M.; Vieil, E. Appl. Biochem. Biotechnol. 2000, 89, 183-193.

(12) Korri-Youssoufi, H.; Garnier, F.; Srivastava, P.; Godillot, P.; Yassar, A. J. Am. Chem. Soc. 1997, 119, 7388-7389.

(13) Livache, T.; Roget, A.; Dejean, E.; Barthet, C.; Bidan, G.; Teoule, R. Nucleic Acids Res. 1994, 22, 2915-2921.

(14) Livache, T.; Fouque, B.; Roget, A.; Marchand, J.; Bidan, G.; Teoule, R.; Mathis, G. Anal. Biochem. 1998, 255, 188-194.

(15) Caruana, D. J.; Heller, A. J. Am. Chem. Soc. 1999,121, 769-774.

(16) Trnka, T. M.; Grubbs, R. H. Acc. Chem. Res. 2001, 34, 18-29.

(17) Watson, K. J.; Zhu, J.; Nguyen, S. T.; Mirkin, C. A. J. Am. Chem. Soc. 1999, 121, 462-463.

(18) Schwab, P.; France, M. B.; Ziller, J. W.; Grubbs, R. H. Angew. Chem., Int. Ed. Engl. 1995, 34, 2039-2041.

(19) Posner, G. H.; Ting, J. S.; Lentz, C. M. Tetrahedron 1976, 32, 2281-7.

(20) Davies, D. I.; Pearce, D. J. A.; Dart, E. C. J. Chem. Soc., Perkin Trans. 1 1973, 433-8.

(21) Storhoff, J. J.; Elghanian, R.; Mucic, R. C.; Mirkin, C. A.; Letsinger, R. L. J. Am. Chem. Soc. 1998, 120, 1959-1964.

(22) Watson, K. J.; Park, S. -J.; Nguyen, S. T.; Mirkin, C. A. J. Am. Chem. Soc. 2001, 123, 5592-5593.

(23) Kelly, S. O.; Boon, E. M.; Barton, J. K.; Jackson, N. M.; Hill, M. G. Nucleic Acids Res. 1999, 27, 4830-4837.

(24) Napier, M. E.; Loomis, C. R.; Sistare, M. F.; Kim, J.; Eckhardt, A. E.; Thorp, H. H. Bioconjugate Chem. 1997, 8, 906-913.

(25) Yu, C. J.; Wan, Y.; Yowanto, H.; Li, J.; Tao, C. L.; James, M. D.; Tan, C. L.; Blackburn, G. F.; Meade, T. J. J. Am. Chem. Soc. 2001, 123, 11155-11161

(26) Shchepinov, M. S.; Mir, K. U.; Elder, J. K.; Frank-Kamenetskii, M. D.; Southern, E. M. Nucleic Acids Res. 1999, 27, 3035-3041.

(27) Storhoff, J. J.; Elghanian, R.; Mucic, R. C.; Mirkin, C. A.; Letsinger, R. L. J. Am. Chem. Soc. 1998, 120, 1959-1964.

(28) Taton, T. A.; Mirkin, C. A.; Letsinger, R. L. Science 2000, 289, 1757.

What we claim:

1. A ROMP polymer or co-polymer having oligonucleotides bound thereto.

2. The ROMP polymer or co-polymer of claim 1 wherein the ROMP co-polymer comprises a ROMP block co-polymer or random co-polymer.

3. The ROMP polymer or co-polymer of claim 1 wherein the ROMP block co-polymer comprises a ROMP multiblock co-polymer.

4. The ROMP polymer or co-polymer of claim 1 wherein the oligonucleotide comprises a spacer portion and a recognition portion wherein the spacer portion is bound to the ROMP polymer, and the recognition portion having a sequence that is complementary to at least one portion of the sequence of another oligonucleotide.

5. The ROMP polymer or co-polymer of claim 1, wherein the oligonucleotides comprise at least one type of recognition oligonucleotides, each type of recognition oligonucleotides comprising a spacer portion and a recognition portion wherein the spacer portion is attached to the ROMP polymer and the recognition portion has a sequence complementary to at least one portion of the sequence of another oligonucleotide.

6. The ROMP polymer or co-polymer of claim 5 wherein the spacer portion comprises from about 4 to about 30 nucleotides.

7. The ROMP polymer or co-polymer of claim 5 wherein the spacer portion comprises about 10 nucleotides.

8. The ROMP polymer or co-polymer of claim 5 wherein the spacer portion comprises at least about 4 nucleotides.

9. The ROMP polymer or co-polymer of claim 5 wherein the bases of the nucleotides of the spacer portion are all adenines, all thymines, all cytosines, all uracils or all guanines.

10. The ROMP polymer or co-polymer of claim 1, wherein the ROMP polymer or co-polymer is derived from the polymerization of at least one monomer that can be polymerized through ring-opening metathesis polymerization in the presence of a metathesis catalyst.

11. The ROMP polymer or co-polymer of claim 1, wherein the ROMP co-polymer is derived from the stepwise polymerization of two or more different monomers that can be polymerized through ring-opening metathesis polymerization in the presence of a metathesis catalyst.

12. The ROMP polymer or co-polymer of claim 1, wherein the ROMP polymer is derived from the simultaneous polymerization of two or more different monomers that can be polymerized through ring-opening metathesis polymerization in the presence of a metathesis catalyst.

13. The ROMP polymer or co-polymer of claims 10-12 wherein the metathesis catalyt comprises a ruthenium or osmium carbene catalyst.

14. The ROMP polymer or co-polymer of claim 10-12 wherein the metathesis catalyst comprises Cl₂Ru(PCy3)₂═CHPh.

15. The ROMP polymer or co-polymer of claim 10-12 wherein the monomer comprises a cyclic mono-olefin.

16. The ROMP polymer or co-polymer of claim 10-12 wherein the monomer comprises a substituted norbornene.

17. The ROMP polymer or co-polymer of claim 16 wherein the substituted norbornene comprises a norbornenyl-modified alcohol.

18. The ROMP polymer or co-polymer of claim 17 wherein the norbornenyl-modified alcohol comprises monomer 2.

19. The ROMP polymer or co-polymer of claim 16 where the substituted norbornene comprises a norbornenyl group modified with an electrochemical tag.

20. The ROMP polymer or co-polymer of claim 16 wherein the substituted norbornene comprise norbornenyl-modified ferrocene.

21. The ROMP polymer or co-polymer of claim 20 wherein the ferrocene-modified norbornene has the following formula: norbornene—linker—ferrocene.

22. A ROMP polymer comprising an oligonucleotide-modified product produced by the ROMP polymerization of monomer 2 to produce a homopolymer template and post-polymerization modification of the polymer template to attach oligonucleotides.

23. A ROMP co-polymer having oligonucleotides bound thereto produced by the process of (a) sequential block ROMP polymerization of monomer 2 and at least one or more different monomers to produce a ROMP co-polymer template; (b) post-polymerization modification of the template, followed by coupling of oligonucleotides to the modified template.

24. The ROMP co-polymer of claim 23 wherein the at least one or more different monomers comprise a substituted norbornene.

25. The ROMP co-polymer of claim 24 wherein the substituted norbornene comprises a norbornenyl group modified with an electrochemical tag.

26. The ROMP co-polymer of claim 24 wherein the substituted norbornene comprises a norbornenyl-substituted ferrocene.

27. Materials or structures comprising a ROMP polymer or co-polymer having oligonucleotides bound thereto.

28. Materials or structures comprising a first and second ROMP polymers or copolymers having oligonucleotides bound thereto, the oligonucleotides bound to the first ROMP polymer or co-polymer having a sequence that is complementary to the oligonucleotides bound to the second ROMP polymer or co-polymer.

29. Materials or structures comprised of:
(a) particles having oligonucleotides attached thereto; and
(b) a connector for holding the particles together, the connector comprising a ROMP polymer or co-polymer having oligonucleotides bound thereto, the oligonucleotides bound to the ROMP polymer or co-polymer having a sequence complementary to at least a portion of the sequence of the oligonucleotides bound to the particles.

30. The materials or structures of claim 29 wherein the oligonucleotides bound to the particles have a spacer portion for attaching the oligonucleotides to the particles and a recognition portion that has a sequence that is complementary to at least a portion of the sequence of another oligonucleotide.

31. Materials or structures comprised of:
(a) particles having oligonucleotides attached thereto, the oligonucleotides comprising at least one type of recognition oligonucleotides, each type of recognition oligonucleotides comprising a spacer portion and a recognition portion, the spacer portion having a functional group through which the spacer portion is bound to the particles, the recognition portion having a sequence complementary to at least one portion of the sequence of another oligonucleotide; and
(b) a connector for holding the particles together, the connector comprising a ROMP polymer or co-polymer having oligonucleotides bound thereto, the oligonucleotides comprise a spacer portion and a recognition portion, wherein the spacer portion is bound to the ROMP polymer or co-polymer and the recognition portion has a sequence complementary to at least one portion of the sequence of the oligonucleotides bound to the particles.

32. Materials or structures comprised of:
(a) particles having oligonucleotides attached thereto, the oligonucleotides comprising:
(i) at least one type of recognition oligonucleotides, each type of recognition oligonucleotides comprising a spacer portion and a recognition portion, the spacer portion having a functional group through which the spacer portion is bound to the particles, the recognition portion having a sequence complementary to at least one portion of the sequence of another oligonucleotide; and
(ii) a type of diluent oligonucleotides; and
(b) a connector for holding the particles together, the connector comprising a ROMP polymer or co-polymer having oligonucleotides bound thereto, the oligonucleotides comprise a spacer portion and a recognition portion, wherein the spacer portion is bound to the ROMP polymer or co-polymer and the recognition portion having a sequence complementary to at least one portion of the sequence of the oligonucleotides bound to the particles.

33. The materials or structures of any one of claims 29-32 wherein the particles are metallic particles, semiconductor particles, polymer latex particles, inorganic particles, insulator particles, or a combination thereof.

34. The materials or structures of claim 33 wherein the metallic particles are made of gold, and the semiconductor particles are made of CdSe/ZnS (core/shell).

35. The materials or structures of claim 33 wherein the polymer latex particles are made of polyacrylates and the inorganic particles are made of silica or metal oxide.

36. The materials or structures of any one of claims 29-32 wherein the ROMP co-polymer comprises a ROMP block co-polymer or ROMP random co-polymer.

37. The materials or structures of claim 36 wherein the ROMP block co-polymer comprises a ROMP multiblock co-polymer.

38. The materials or structures of claim 29-32 wherein the spacer portion of the oligonucleotides bound to the ROMP polymer or co-polymer comprises from about 4 to about 30 nucleotides.

39. The materials or structures of claim 38 wherein the spacer portion comprises at least 10 nucleotides.

40. The materials or structures of any one of claims 29-32 wherein the spacer portion comprises at least 4 nucleotides.

41. The materials or structures of claim 40 wherein the spacer portion of the oligonucleotides bound to the particles comprises from about 10 to about 30 nucleotides.

42. The materials or structures of claim 40 wherein the spacer portion comprises at least 10 nucleotides.

43. The materials or structures of any one of claims 29-32 wherein the bases of the nucleotides of the spacer portion are all adenines, all thymines, all cytosines, all uracils or all guanines.

44. The materials or structures of claim 32 wherein the diluent oligonucleotides contain about the same number of nucleotides as are contained in the spacer portions of the recognition oligonucleotides.

45. The materials or structures of claim 44 wherein the sequence of the diluent oligonucleotides is the same as that of the spacer portions of the recognition oligonucleotides.

46. Materials or structures comprised of:
(a) at least two types of particles having oligonucleotides attached thereto, the first type of particle having at least two types of oligonucleotides, the first type of oligonucleotides bound to the first type of particles having a sequence that is complementary to at least a portion of the sequence of the oligonucleotides bound to a second type of particle; and
(b) oligonucleotide polymer conjugates for holding the particles together, the oligonucleotide polymer conjugate comprising a ROMP polymer having oligonucleotides bound thereto, the oligonucleotides of the oligonucleotide polymer conjugate having a sequence complementary to at least one portion of the sequence of a second type of oligonucleotides bound to the first type of particles.

47. The materials or structures of claim 46 wherein the particles are metallic particles, semiconductor particles, polymer latex particles, insulator particles, inorganic particles or a combination thereof.

48. The materials or structures of claim 47 wherein the metallic particles are made of gold, and the semiconductor particles are made of CdSe/ZnS (core/shell).

49. The materials or structures of claim 47 wherein the polymer latex particles are made of polyacrylates, and the inorganic particles or insulator particles are made silica or metal oxide.

50. The materials or structures of claim 46 wherein the ROMP co polymer comprises a ROMP block co-polymer or ROMP random co-polymer.

51. The materials or structures of claim 50 wherein the ROMP block co-polymer comprises a ROMP multiblock co-polymer.

52. A method of fabrication comprising
providing a ROMP polymer or co-polymer having at least one type of oligonucleotides bound thereto, the oligonucleotides having a selected sequence, the sequence of each type of oligonucleotide having at least two portions;
providing one or more types of particle-oligonucleotide conjugates, the oligonucleotides attached to the particles of each of the types of conjugates having a sequence complementary to the sequence of a portion of a oligonucleotide bound to the ROMP polymer or co-polymer; and
contacting the ROMP polymer or co-polymer and particle oligonupcleotide conjugates under conditions effective to allow hybridization of the oligonucleotides attached to the particles to the oligonucleotides bound to the ROMP polymer or co-polymer so that a desired material or structure is formed wherein the particles conjugates are held together by oligonucleotides bound to the ROMP polymer.

53. A method of fabrication comprising:
providing at least two types of particle-oligonucleotide conjugates, the first type of particle-oligonucleotide conjugates have at least two types of oligonucleotides wherein the first type of oligonucleotides attached to the first type of particle-oligonucleotide conjugates has a sequence that is complementary to that of the oligonucleotides attached to the particles of the second type of conjugates, the second type of oligonucleotides attached to the particles of the first type of conjugates having a sequence that is complementary to that of the oligonucleotides attached to the particles of a second type of conjugates;
providing a ROMP polymer or co-polymer having oligonucleotides bound thereto, the oligonucleotides having a sequence that is complementary to a second type of oligonucleotides bound to the first type of particle-oligonucleotide conjugates;
contacting the first and second types of particle-oligonucleotide conjugates with the ROMP polymer or co-polymer under conditions effective to allow hybridization of the oligonucleotides on the first type of particle-oligonucleotide conjugates with the oligonucleotides on the second type of particle-oligonucleotide conjugates and on the ROMP polymer or co-polymer so that a desired material or structure is formed.

54. A method for preparing a ROMP polymer or co-polymer having oligonucleotides bound thereto, the method comprising:
providing (i) a ROMP polymer or co-polymer modified with chlorophosphoramidite and (ii) oligonucleotides bound to a solid support;
contacting the chlorophosphoramidite-modified ROMP polymer with the oligonucleotides bound to a support to produce an oligonucleotide ROMP polymer conjugate bound to the support; and
cleaving the oligonucleotide-modified ROMP polymer or copolymer from the support.

55. A method of fabrication comprising:
providing first and second ROMP polymers or co-polymers having oligonucleotides bound thereto, the oligonucleotides bound to the first ROMP polymer or co-polymer having a sequence that is complementary to the oligonucleotides bound to the second ROMP polymer or co-polymer; and
contacting the first and second ROMP polymers or co-polymer under conditions effective to allow hybridization of the oligonucleotides on the first ROMP polymer or co-polymer with the oligonucleotides on the second ROMP polymer or co-polymer so that a desired material or structure is formed.

56. The methods of claims 52-55 wherein the ROMP co-polymer comprises a ROMP block co-polymer or ROMP random co-polymer.

57. In a method for the detection of one or more target nucleic acids in a sample, the sequence of each nucleic acid having at least two portions, the method comprising:
providing one or more types of oligonucleotide-modified ROMP polymer or copolymer, the sequence of the oligonucleotides bound to each type of polymer or copolymer has at least two portions wherein at least one portion of the sequence of the oligonucleotides is complementary to first portion of a sequence of a target nucleic acid, wherein the oligonucleotides bound to one type of polymer or copolymer is different from another type, wherein each type of polymer or copolymer serves as a unique identifier for a particular target nucleic acid, and wherein the polymer or copolymer includes electrochemical labels;
providing a gold electrode surface having oligonucleotides bound thereto, the oligonucleotides that are bound to the surface have a sequence having at least two portions wherein the first portion of the oligonucleotides is complementary to a second portion of the target nucleic acid;
contacting the one or more types of oligonucleotide-modified ROMP polymer or copolymer, the gold surface, and the sample under conditions effective to allow for hybridization of the oligonucleotides bound to the polymer or copolymer with the target nucleic acids and for hybridization of the oligonucleotides bound to the surface with the target nucleic acids to form a complex on the surface in the presence of one or more target nucleic acids; and
electrochemically detecting for the presence of the complex,.

58. The method of claim 57 wherein the ROMP polymer or copolymer are chemically defined and includes a defined number of electrochemical labels.

59. The method of claim 57, wherein the electrochemical detection occurs using cyclic voltammetry or differential pulse voltammetry.

60. The method of claim 57, wherein each type of ROMP polymer or copolymer is a ROMP block co-polymer having different redox.

61. The method according to claim 57, wherein the substrate having plurality of types of oligonucleotides attached thereto in an array to allow for the detection of multiple different nucleic acid targets.

62. The method according to claim 57 wherein the sample is first contacted with the surface so that one or more target nucleic acids hybridizes with complementary oligonucleotides bound to the surface and then the target nucleic acids bound to the surface is contacted with the polymer or copolymer so that at least some of the oligonucleotides bound to the polymer or copolymer hybridize with a portion of the sequence of the target nucleic acid bound to the surface.

63. The method according to claim 57 wherein the polymer or copolymer is contacted with the sample so that at least some of the oligonucleotides bound to the polymer or copolymer hybridize with a portion of the sequence of the target nucleic acids; and contacting the target nucleic acids bound to the polymer or copolymer with the surface so that a portion of the sequence of the target nucleic acids bound to the polymer or copolymer hybridizes with complementary oligonucleotides bound to the surface.

64. The method according to claim 57 wherein the sample, polymer or copolymer, and surface are contacted simultaneously.

65. The method of claim 57 further comprising
providing a second oligonucleotide-modified ROMP polymer or copolymer, the sequence of the oligonucleotides bound to the second polymer or copolymer has at least two portions wherein at least one portion of the sequence of the oligonucleotides bound to the second polymer or copolymer is complementary to oligonucleotides bound to the first oligonucleotide-modified ROMP polymer or co-polymer; and
contacting the second ROMP polymer or co-polymer with the one or more types of the first ROMP polymer or copolymer bound to the surface.

66. The method of claim 65 further comprising
providing a third oligonucleotide-modified ROMP polymer or copolymer, the sequence of the oligonucleotides bound to the second polymer or copolymer has at least two portions wherein at least one portion of the sequence of the oligonucleotides bound to the second polymer or copolymer is complementary to oligonucleotides bound to the first oligonucleotide-modified ROMP polymer or co-polymer; and
contacting the third ROMP polymer or co-polymer with the second ROMP polymer or copolymer bound to the surface.

67. A kit for detecting a target nucleic acid in a sample, the kit comprising at least one or more containers including one or more types of chlorophosphoramidite modified ROMP polymer or copolymer, wherein each polymer or copolymer has a different redox activity and can be used for coupling with oligonucleotides.

68. A kit for detecting a target nucleic acid in a sample, the kit comprising at least one or more containers including one or more types of chlorophosphoramidite-modifiable ROMP polymer or copolymer, wherein each polymer or copolymer has a different redox activity and can serve as an identifier for a specific target nucleic acid.

69. A kit for detecting a target nucleic acid in a sample, the kit comprising at least one or more containers including one or more types of oligonucleotide-modified ROMP polymer or copolymer, wherein each polymer or copolymer has a different redox activity and serves as an identifier for a specific target nucleic acid.

70. A system for detecting one or more target nucleic acids in a sample, the sequence of target nucleic acids have at least two portions, in a sample comprising
(a) one or more types of oligonucleotide-modified ROMP polymer or copolymer, wherein each polymer or copolymer has a different redox activity and serves as an identifier for a specific target nucleic acid, the oligonucleotides bound to one type of polymer or copolymer is different from another, the oligonucleotides have a sequence having at least two portions, one portion of the sequence of the oligonucleotides is complementary to a first portion of a target nucleic acid;
(b) a gold electrode surface having oligonucleotides bound thereto wherein the oligonucleotides bound to the surface has a sequence that is complementary to a second portion of a target nucleic acid; and
(c) a detector for electrochemical detection of one or more polymers or copolymers bound to the surface in the presence of one or more target nucleic acids.
